(19) United States
(12) Patent Application Publication
(10) Pub. No.: US 2026/0199022 A1
(43) Pub. Date: Jul. 16, 2026

(54) SYSTEM AND METHOD OF IMAGE GENERATION FOR TRANS-CATHETER AORTIC VALVE REPLACEMENT GUIDING

(71) Applicant: Libra Science Ltd., Road Town, Tortola (VG)

(72) Inventor: Shlomo BEN-HAIM, Milan (IT)

(73) Assignee: Libra Science Ltd., Road Town, Tortola (VG)

(21) Appl. No.: 19/135,117

(22) PCT Filed: Nov. 29, 2023
(86) PCT No.: PCT/IB2023/062015

§ 371 Date: Jun. 3, 2025

Related U.S. Application Data
(60) Provisional application No. 63/430,063, filed on Dec. 5, 2022.
(60) Provisional application No. 63/466,722, filed on May 16, 2023.
(60) Provisional application No. 63/528,450, filed on Jul. 24, 2023.
(60) Provisional application No. 63/530,724, filed on Aug. 4, 2023.
(60) Provisional application No. 63/531,063, filed on Aug. 7, 2023.

(51) Int. Cl. A61B 34/20; A61B 6/00; A61B 6/50; A61B 90/00; A61F 2/24; A61M 25/01; G06T 7/00; G06T 7/246; G06T 7/73; G06T 11/00; G16H 50/20

(52) U.S. Cl. CPC A61B 34/20; A61B 6/481; A61B 6/487; A61B 6/503; A61F 2/2427; G06T 7/0014; G06T 7/248; G06T 7/74; G06T 11/00; G16H 50/20; A61B 2034/2065; A61B 2090/364; A61M 2025/0166; G06T 2207/10064; G06T 2207/20081; G06T 2207/30021; G06T 2207/30048; G06T 2210/41

(57) ABSTRACT

There is provided a computer implemented method of guiding a trans-catheter aortic valve replacement intervention in a subject, comprising: obtaining at least one fluoroscopic image depicting an aortic valve prosthesis device in the subject, the at least one fluoroscopic image excludes injected contrast, feeding the at least one fluoroscopic image into a machine leaning (ML) model, obtaining an indication of the annulus line of the heart of the subject, and presenting the indication, optionally as an overlay of the annulus line over the at least one fluoroscopic image.

### RELATED APPLICATIONS

This application claims the benefit of priority of U.S. Provisional Patent Application 63/430,063 filed on Dec. 5, 2022, U.S. Provisional Patent Application No. 63/466,722 filed on May 16, 2023, U.S. Provisional Patent Application No. 63/528,450 filed on Jul. 24, 2023, U.S. Provisional Patent Application No. 63/530,724 filed on Aug. 4, 2023, and U.S. Provisional Patent Application No. 63/531,063 filed on Aug. 7, 2023, the contents of which are incorporated herein by reference in their entirety.

### BACKGROUND

The present invention, in some embodiments thereof, relates to aortic valve replacement and, more specifically, but not exclusively, to systems and methods for guiding aortic valve replacement.

Currently, implantable medical devices, such as large stents, scaffolds, and other cardiac intervention devices are utilized to repair or replace problem native biological systems. For example, heart valve replacement in patients with severe valve disease is a common surgical procedure. The replacement can conventionally be performed by open heart surgery, in which the heart is usually arrested and the patient is placed on a heart bypass machine. In recent years, prosthetic heart valves have been developed which are implanted using minimally invasive procedures such as transapical or percutaneous approaches. These procedures involve compressing the prosthetic heart valve radially to reduce its diameter, inserting the prosthetic heart valve into a delivery device, such as a catheter, and advancing the delivery device to the correct anatomical position in the heart. Once properly positioned, the prosthetic heart valve is deployed by radial expansion within the native valve annulus.

### SUMMARY

According to some embodiments of the present invention, there is provided a computer implemented method of generating an image for guiding a trans-catheter aortic valve replacement intervention in a subject, comprising: obtaining at least one fluoroscopic image depicting an aortic valve prosthesis device in the subject, the at least one fluoroscopic image excludes injected contrast, feeding the at least one fluoroscopic image into a machine leaning (ML) model, obtaining an indication of the annulus line of the heart of the subject, and presenting the indication.

Optionally, the at least one fluoroscopic image comprises at least one non-contrast image depicting a structure associated with a valve, and further comprising: obtaining at least one contrast image comprising at least one fluoroscopic image including injected contrast, depicting the annulus line of the heart, obtained at a same zoom, subject orientation, and pose of an image sensor that captured the non-contrast image, feeding a combination of the at least one non-contrast image and the at one contrast image into the machine learning model, wherein the indication of the annulus line is presented as a visual overlay over the at least one non-contrast image.

Optionally, the at least one non-contrast image and the at least one contrast image depict a same fiducial object.

Optionally, the fiducial object comprises a catheter located in the aorta of the subject.

Optionally, the machine learning model is trained on a training dataset of multiple records, each record includes a combination of at least one non-contrast image that depicts the structure associated with the valve and a fiducial object, and at least one contrast image that depicts the annulus line of the heart of the subject and the fiducial object.

Optionally, the method further comprises detecting a change in at least one of the zoom, subject orientation, and the pose of the image sensor, and feeding the detected change to the machine learning model in combination with the non-contrast image.

Optionally, further comprising feeding pairs of sequentially obtained non-contrast images into the machine learning model for tracking a location of the annulus line.

Optionally, further comprising presenting the indication as a visual overlay over the at least one fluoroscopic image on a display.

Optionally, the machine learning model is trained on a training dataset of fluoroscopic images that depict the annulus line of the heart of the subject, and a ground truth of the indication for the annulus line of the heart of the subject.

Optionally, the obtaining the at least one fluoroscopic image, the feeding, the obtaining the indication, and the presenting, are iterated while the valve is transported on its way to be implanted in a native aortic annulus.

Optionally, the method further comprises processing the at least one fluoroscopic image by comparing the fluoroscopic angulation of the at least one fluoroscopic image to at least one image of a training dataset used to train the ML model and/or at least one image captured prior to the procedure that excludes the aortic valve prosthesis device.

Optionally, the indication comprises at least one of: position of the annulus line of the heart of the subject and orientation of the annulus line of the heart of the subject.

Optionally, the method further comprises extracting a plurality of landmarks from the at least one fluoroscopic image, and feeding the plurality of landmarks into the ML model to obtain the indication.

Optionally, the plurality of landmarks includes one of more of: anatomical structures around the annulus line and signature of calcification presence in the heart.

Optionally, the ML model is a generic ML model, and the method further comprises dynamically updating the generic ML model to create a customized ML model for the subject by dynamically creating customized records that include fluoroscopic images of the subject, and dynamically training the generic ML model on the customized records.

Optionally, the customized records that include fluoroscopic images of the subject were taken when the subject was injected with contrast.

Optionally, the customized records include a first set of fluoroscopic images of the subject with contrast and a second set of fluoroscopic images without contrast, wherein ground truth labels for a target anatomical feature are applied to the customized records with the first set of fluoroscopic images with contrast.

According to some embodiments of the present invention, there is provided a computer implemented method for training a machine learning model for guiding a trans-catheter aortic valve replacement intervention in a subject, comprising: creating a training dataset that includes a plurality of records, wherein a record includes: at least one of: at least one fluoroscopic image depicting an aortic valve prosthesis device in an aorta of the subject that excludes injected contrast, and at least one fluoroscopic image that includes injected contrast, a ground truth label of an indication of the annulus line of the heart of the subject, and training the ML model on the training dataset.

Optionally, the at least one fluoroscopic image that excludes injected contrast and the at least one fluoroscopic image that includes injected contrast, depict a same fiducial object.

Optionally, the at least one fluoroscopic image that excludes injected contrast depicts a structure associated with a valve, and the at least one fluoroscopic image that includes injected contrast depicts the annulus line.

Optionally, the training dataset includes images of the subject acquired prior to the procedure that exclude the aortic valve prosthesis.

Optionally, the images of the subject acquired prior to the procedure include one or more of: CT images and MRI images.

Optionally, the images of the subject acquired prior to the procedure include signature of calcification presence in the heart of the subject.

Optionally, the training dataset includes images of a plurality of subjects, and the ML model comprises a generic ML model, and the method further comprises creating at least one customized record for a certain subject that includes images of the certain subject, and converting the generic ML model to a customized ML model by applying a transfer learning approach by training the generic ML model on the at least one customized record.

Optionally, the method further comprises extracting at least one landmark feature from the at least one fluoroscopic image, and including the at least one landmark feature in the record.

Optionally, the at least one landmark feature includes signature of calcification presence in the heart of the subject.

According to some embodiments of the present invention, there is provided a system for generating an image for guiding a trans-catheter aortic valve replacement intervention in a subject, comprising: at least one processor executing a code for: obtaining at least one fluoroscopic image depicting an aortic valve prosthesis device in the subject, the at least one fluoroscopic image excludes injected contrast, feeding the at least one fluoroscopic image into a machine leaning model, obtaining an indication of the annulus line of the heart of the subject, and presenting the indication as a visual overlay over the at least one fluoroscopic image on a display.

According to some embodiments of the present invention, there is provided a system for training a machine learning model for guiding a trans-catheter aortic valve replacement intervention in a subject, comprising: at least one processor executing a code for: creating a training dataset that includes a plurality of records, wherein a record includes: at least one of: at least one fluoroscopic image depicting an aortic valve prosthesis device in the subject that excludes injected contrast, and the at least one fluoroscopic image that includes injected contrast, a ground truth label of an indication of the annulus line of the heart of the subject, and training the ML model on the training dataset.

According to some embodiments of the present invention, there is provided a computer implemented method of guiding a transcatheter aortic valve replacement, comprising: accessing a machine learning (ML) model trained on a training dataset of a plurality of records, each record including a sample image of a sample individual or of a generic model labelled with a ground truth label indicating at least one of a lower annulus of a native aortic valve, dynamically further training the machine learning (ML) model on at least one image of a subject depicting the lower annulus of the native aortic valve, to obtain a personalized ML model, dynamically feeding at least one target image of the subject into the personalized ML model to obtain a detected lower annulus, and displaying the detected lower annulus of the subject over the target image.

Optionally, the at least one image of a subject depicting the lower annulus of the native aortic valve is a CT or MRI image.

Optionally, the at least one image of a subject depicting the lower annulus of the native aortic valve is a fluoroscopic image taken when the subject was injected with contrast.

Optionally, the at least one target image of the subject is a fluoroscopic image.

According to some embodiments of the present invention, there is provided a computer implemented method of guiding a medical intervention, comprising: automatically detecting a structure within a native valve of a subject by analyzing a target image that excludes injected contrast, and presenting the detected structure within a native valve of a subject as a visual overlay over the target image on a display.

Optionally, the structure comprises a first structure, and the method further comprises: automatically analyzing a contrast image that includes injected contrast to detect a second structure associated with the native valve, and computing a mapping between the first structure and the second structure, wherein automatically detecting the first structure comprises: analyzing the target image to detect the second structure, and detecting the first structure by applying the mapping to the detected second structure.

Optionally, the target image depicts a fiducial object, and the method further comprises: computing a first sub-mapping between the first structure and the fiducial object, computing a second sub-mapping between the second structure and the fiducial object, computing the mapping between the first structure and the second structure according to a combination of the first sub-mapping and the second sub-mapping.

Optionally, at least one of: the first structure comprises a calcification signature of calcium deposits on the valve, the fiducial object comprises a catheter located in the aorta, and the second structure comprises a contour of an annulus of the native valve.

Optionally, the structure comprises an annulus of the native valve, and the method further comprises: automatically analyzing a contrast image that includes injected contrast to detect the annulus of the native valve, computing a spatial relationship between the annulus and a detected fiducial object, wherein automatically detecting the structure comprises: analyzing the target image that excludes contrast to detect the fiducial object, and detecting the annulus by applying the spatial relationship to the detected fiducial object.

Optionally, the annulus is detected by detecting at least two nadirs of at least two cusps of a native valve in the contrast image, and defining a line though the at least two nadirs.

Optionally, the fiducial object comprises an accessory catheter located in proximity to the native valve, wherein the spatial relationship is between the annulus and a center of mass of the accessory catheter.

Optionally, the annulus detected on the contrast image comprises a pre-event annulus prior to an event that disrupts the annulus, and the method further comprises detecting the annulus after the event that disrupts the annulus according to the spatial relationship computed prior to the event.

Optionally, the event comprises inflation of a balloon within the native valve.

Optionally, the native valve is a native aortic valve.

Optionally, the structure within the native valve is a lower annulus of a subject's native aortic valve.

Optionally, the medical intervention is a transcatheter aortic valve replacement procedure.

Optionally, the native valve is a tricuspid valve.

Optionally, the native valve is a mitral valve.

Optionally, the native valve is a pulmonary valve.

Optionally, automatically detecting includes feeding the target image into a machine leaning model.

Optionally, the target image is a fluoroscopic image.

Optionally, automatically detecting includes processing the target image by cross correlation the target image with template of calcification signature of the subject.

Optionally, the template of calcification signature of the subject is calculated from CT or MRI image of the subject.

Optionally, the template of calcification signature of the subject is calculated from fluoroscopic image of the subject taken when the subject was injected with contrast.

Optionally, the automatically detecting and the presenting, are iterated while the valve prosthesis device is transported on its way to be implanted in a native valve.

According to some embodiments of the present invention, there is provided a system for guiding a medical intervention comprising: at least one processor executing a code for: automatically detecting a structure within a native valve of a subject by analyzing a target image depicting a valve prosthesis device in the subject, the target image excludes injected contrast, and presenting the detected structure within a native valve of a subject as a visual overlay over the target image on a display.

Optionally, the structure comprises a first structure, and further comprising code for: automatically analyzing a contrast image that includes injected contrast to detect a second structure associated with the native valve, and computing a mapping between the first structure and the second structure, wherein automatically detecting the first structure comprises: analyzing the target image to detect the second structure, and detecting the first structure by applying the mapping to the detected second structure.

Optionally, the target image depicts a fiducial object, and further comprising code for: computing a first sub-mapping between the first structure and the fiducial object, computing a second sub-mapping between the second structure and the fiducial object, computing the mapping between the first structure and the second structure according to a combination of the first sub-mapping and the second sub-mapping.

Optionally, at least one of: the first structure comprises a calcification signature of calcium deposits on the valve, the fiducial object comprises a catheter located in the aorta, and the second structure comprises a contour of an annulus of the native valve.

Optionally, including the display.

Optionally, the native valve is a native aortic valve.

Optionally, the structure within the native valve is a lower annulus of a subject's native aortic valve.

Optionally, the medical intervention is a transcatheter aortic valve replacement procedure.

Optionally, the native valve is a tricuspid valve.

Optionally, the native valve is a mitral valve.

Optionally, the native valve is a pulmonary valve.

Optionally, automatically detecting includes feeding the target image into a machine leaning model.

Optionally, the target image is a fluoroscopic image.

Optionally, automatically detecting includes processing the target image by cross correlation the target image with template of calcification signature of the subject.

Optionally, the template of calcification signature of the subject is calculated from CT or MRI image of the subject.

Optionally, the template of calcification signature of the subject is calculated from fluoroscopic image of the subject taken when the subject was injected with contrast.

Optionally, the automatically detecting and the presenting, are iterated while the valve prosthesis device is transported on its way to be implanted in a native valve.

According to some embodiments of the present invention, there is provided a computer implemented method of guiding a medical intervention in a subject, comprising: automatically detecting a first anatomical structure in a target image, automatically co-locating said first anatomical structure to a second anatomical structure, and presenting the co-located second structure as a visual overlay over the target image on a display.

Optionally, the method further comprises computing a first mapping between the first anatomical structure and a fiducial object depicted in a non-contrast image, computing a second mapping between the second anatomical structure the fiducial object depicted in a contrast enhanced image, computing a combined mapping as a combination of the first mapping and the second mapping, wherein co-locating comprises applying the combined mapping to the detected first anatomical structure.

Optionally, the first mapping and the second mapping are computed for a pair of the non-contrast image and the contrast enhanced image captured at a same pose of an image sensor, and subject orientation, and zoom.

Optionally, the first anatomical structure is calcification signature.

Optionally, the first anatomical structure is tissue thickening.

Optionally, the first anatomical structure is blood flow derived dynamic changes.

Optionally, automatically co-locating said first anatomical structure to a second anatomical structure is based on an identified spatial relationship between the first and second anatomical structures.

Optionally, the spatial relationship between the first and second anatomical structures is calculated from CT or MRI image of the subject taken prior to the intervention.

Optionally, the spatial relationship between the first and second anatomical structures is calculated from fluoroscopic image of the subject taken when the subject was injected with contrast during the intervention.

Optionally, the second anatomical structure is a structure within a native valve of the subject.

Optionally, the target image depicts a valve prosthesis device in the subject.

Optionally, the native valve is a native aortic valve.

Optionally, the structure within the native valve is selected from a group comprising: a lower annulus of a subject's native aortic valve, middle of a subject's native aortic valve, top of a subject's native aortic valve, one or more commissures of a subject's native aortic valve, the leaflets of a subject's native aortic valve, the sinuses next to a subject's native aortic valve.

Optionally, the medical intervention is a transcatheter aortic valve replacement procedure.

Optionally, the native valve is a tricuspid valve.

Optionally, the native valve is a mitral valve.

Optionally, the native valve is a pulmonary valve.

Optionally, the target image was taken when no contrast was injected.

Optionally, automatically detecting the first anatomical structure includes feeding the target image into a machine leaning model.

Optionally, the target image is a fluoroscopic image.

Optionally, automatically detecting the first anatomical structure in the target image includes processing the target image by cross correlation the target image with template of the first anatomical structure.

According to some embodiments of the present invention, there is provided a system for guiding a medical intervention comprising: at least one processor executing a code for: automatically detecting a first anatomical structure in a target image, automatically co-locating said first anatomical structure to a second anatomical structure, and presenting the co-located second structure as a visual overlay over the target image on a display.

According to some embodiments of the present invention, there is provided a computer implemented method of guiding a transcatheter aortic valve replacement, comprising: automatically detecting a lower annulus of a subject's native aortic valve by analyzing a target image depicting a partial expansion of a transcatheter heart valve (THV) positioned within the native aortic valve of the subject, automatically detecting a distal end of the partially expanded THV by analyzing the target image, computing a distance between the lower annulus and the distal end, and providing the distance over the target image.

Optionally, the method further comprises determining whether the distance is within a target range indicating correct positioning of the THV within the native aortic annulus.

Optionally, automatically detecting comprises feeding the target image into a machine learning model trained on a training dataset of a plurality of records, each record including a sample image of the partially expanded THV, and a ground truth indicating the distal end thereof.

Optionally, the method further comprises when the distance is within the target range, treating the subject for a dysfunctional aortic valve by implanting the THV within the native aortic valve.

Optionally, the method further comprises when the distance is external to the target range, moving the THV to another location.

Optionally, detecting the lower annulus comprises computing a first line representing a plane of the lower annulus, wherein detecting the distal end comprises computing a second line representing a plane of the distal end of partially expanded THV, and wherein computing the distance comprises computing the distance between the first line and the second line.

Optionally, the THV comprises a self-expanding THV, and the method further comprises monitoring the distance over a plurality of sequential target images during the self-expansion of the THV.

Optionally, the method further comprises automatically detecting a fiducial marker of the THV having a known dimension, and computing a calibrated distance by calibrating the distance according to the known dimension, wherein determining comprises determining whether the calibrated distance is within the target range.

Optionally, the fiducial marker comprises a strut or portion thereof of a scaffold of the THV that remains the known fixed dimension throughout expansion of the THV.

Optionally, the method further comprises generating an overlay over the target image that visually marks at least one of the lower annulus and the distal end.

Optionally, the method further comprises generating at least one of a visual and an audio indication of when the distance is within the target range.

Optionally, the method further comprises when no distal end of the partially expanded THV is detected, generating at least one of a visual and an audio indication of where to position a distal end of the THV in the compressed state for expansion of the THV such that the distal end of the THV when expanded is predicted to be located within the target range.

Optionally, the target range is defined according to the an identifier of the THV, wherein different THV have different identifiers and different target ranges.

Optionally, the method further comprises detecting a candidate location of a lower annulus of a native aortic valve by analyzing at least one sample image depicting a lower annulus of a native aortic valve that excludes presence of a transcatheter heart valve (THV) within the native aortic valve, wherein the automatically detecting is further according to the detected candidate location.

Optionally, the candidate location is learned from the at least one sample images of the subject obtained prior to placing the THV in the native aortic valve.

Optionally, the candidate location is learned from at least one sample image of at least one sample individual and/or of a generic model.

Optionally, the candidate location and the lower annulus are detected and tracked over a plurality of sequential sample images and target images by matching phases of at least one breathing cycle and/or at least one cardiac cycle depicted in the sample images to corresponding phases of the target images.

Optionally, automatically detecting the lower annulus by analyzing the target image comprises registering the at least one sample image with the target image, and mapping the candidate location of the at least sample image to the target image to obtain the lower annulus of the target image.

Optionally, mapping comprises computing a transformation from the sample image to the target image, and applying the transformation to the candidate location for obtaining the lower annulus.

Optionally, the at least one sample image includes injected contrast located within the cusps of the aortic valve and the ascending aorta, and the target image excludes injected contrast within the cusps of the aortic valve and the ascending aorta, wherein the lower annulus detected in the at least one sample image is mapped to the target image for identifying the lower annulus in the target image.

According to some embodiments of the present invention, there is provided a computer implemented method of guiding a transcatheter aortic valve replacement, comprising: automatically detecting a lower annulus of a subject's native aortic valve by analyzing a target image for automatically detecting at least one landmark depicted in the target image, and projecting the at least one landmark to the lower annulus for inferring the lower annulus according to the projection.

Optionally, the at least one landmark comprises a catheter placed within an aorta in proximity to the native aortic valve.

Optionally, the at least one landmark is selected from: a feature of the native aortic valve, a feature of the left ventricular outflow tract (LVOT), a feature of a coronary artery.

Optionally, the method further comprises iteratively correcting the lower annuls on subsequent target images based on impact of changes of orientation of an image sensor capturing the subsequent target images on the projection.

As used herein, the phrases signature of calcification presence and calcification signature, are interchangeable.

According to some embodiments of the present invention, there is provided a computer implemented method of guiding a transcatheter aortic valve replacement, comprising: obtaining a 2D target image depicting a native valve of a subject, detecting a calcification signature in the 2D target image, selecting a calcification template from a plurality of calcification templates of a 3D image of the subject according to a pose of the image sensor that captured the 2D target image, wherein an annulus plane is indicated on the 3D image, registering the 2D target image to the calcification template, wherein the annulus plane of the 3D image is mapped to an annulus line on the calcification template, and mapping the annulus line on the calcification template to the registered 2D target image.

Optionally, the calcification template is computed by projecting the 3D image with the annulus plane to a 2D plane having the pose of the image sensor.

Optionally, the calcification template is computed by searching the calcification signature for a 2D calcification template of the 3D image according to a correlation requirement.

Optionally, the method further comprises iteratively tracking the annulus line on subsequent images by dynamically correcting for the pose of the image sensor.

Optionally, the calcification signature is identified on an anatomical structure that shares similar displacement during cardiac and/or breathing cycles with the annulus line.

Optionally, the calcification signature is identified in tissues that are not significantly impacted by a balloon inflated within the native valve.

Optionally, the automatically detecting the lower annulus and automatically detecting the distal end comprises: accessing a machine learning (ML) model trained on a training dataset of a plurality of records, each record including a sample image of a sample individual or of a generic model labelled with a ground truth label indicating at least one of a lower annulus of a native aortic valve and a distal end of a partial expanded transcatheter heart valve (THV) for positioning within the native aortic valve, dynamically further training the machine learning (ML) model on at least one image of a subject depicting the lower annulus of the native aortic valve, to obtain a personalized ML model, and dynamically feeding at least one target image of the subject into the personalized ML model to obtain the detected lower annulus and the detected distal end of the partially expanded THV.

Optionally, the at least one image of the subject used to train the ML model comprises a plurality of images captured over a plurality of phases of a cardiac cycle and/or a breathing cycle of the subject.

Optionally, the method further comprises computing a personalized mask excluding a region around the lower annulus and the distal end of the partially expanded THV, and applying the mask to the sample image of the record used to train the ML model and/or to the at least one target image fed into the ML model.

Optionally, the method further comprises analyzing at least one contrast enhanced image of the subject depicting injected contrast located within the cusps of the aortic valve and the ascending aorta, to identify the lower annulus, automatically labelling the identified lower annulus in the at least one contrast enhanced image, generating a mask over the contrast enhanced region of the contrast enhanced image that includes the cusps and the ascending aorta to define a masked region, creating a synthesized image by replacing the masked region of the contrast enhanced region with values of pixels corresponding to the masked region in a non-contrast enhanced image, and providing the record comprising the synthesized image labeled with the identified lower annulus for training the ML model for detecting the lower annulus in response to an input of a non-contrast enhanced image.

According to some embodiments of the present invention, there is provided a computer implemented method of guiding a transcatheter aortic valve replacement, comprising: automatically detecting a lower annulus of a subject's native aortic valve by analyzing a target image depicting a partial expansion of a transcatheter heart valve (THV) positioned within the native aortic valve of the subject, feeding the target image into a ML model trained on a training dataset of sample images of partially expanded THV labelled with a ground truth indicating the distal end thereof, obtaining a detected distal end of the partially expanded THV as an outcome of the ML model, computing a distance between the lower annulus and the distal end, and providing the distance over the target image.

Optionally, the detection of the lower annulus is obtained as an outcome of feeding the target image into a second ML model trained on a training dataset of sample images of partially expanded THVs labelled with a ground truth indicating the lower annulus.

Optionally, the lower annulus is detected by mapping from a catheter located in the aorta.

According to some embodiments of the present invention, there is provided a computer implemented method of guiding a transcatheter aortic valve replacement, comprising: accessing a machine learning (ML) model trained on a training dataset of a plurality of records, each record including a sample image of a sample individual or of a generic model labelled with a ground truth label indicating at least one of a lower annulus of a native aortic valve and a distal end of a partial expanded transcatheter heart valve (THV) for positioning within the native aortic valve, dynamically further training the machine learning (ML) model on at least one image of a subject depicting the lower annulus of the native aortic valve, to obtain a personalized ML model, dynamically feeding at least one target image of the subject into the personalized ML model to obtain a detected lower annulus and a detected distal end of the partially expanded THV, computing a distance between the lower annulus and the distal end, and determining when the distance is within a target range indicating correct positioning of the THV within the native aortic annulus.

According to some embodiments of the present invention, there is provided a computer implemented method of guiding a transcatheter aortic valve replacement, comprising: automatically detecting a lower annulus of a subject's native aortic valve by analyzing a first target image that excludes a transcatheter heart valve (THV) positioned within the native aortic valve of the subject, automatically detecting a distal end of a partially expanded THV positioned within the native aortic valve of the subject by analyzing a second target image, mapping the lower annulus of the first target image to the second target image, computing a distance between the lower annulus of the second target image and the distal end detected in the second target image, and determining whether the distance is within a target range indicating correct positioning of the THV within the native aortic annulus.

Optionally, the first image includes contrast depicted in the aortic cusps and the ascending aorta and the second image excludes contrast in the aortic cusps and the ascending aorta.

According to some embodiments of the present invention, there is provided a computer implemented method of guiding a transcatheter aortic valve replacement, comprising: automatically detecting a lower annulus of a subject's native aortic valve by analyzing a first target image that excludes a transcatheter heart valve (THV) positioned within the native aortic valve of the subject, automatically detecting a distal end of a partially expanded THV positioned within the native aortic valve of the subject by analyzing a second target image, mapping the lower annulus of the first target image to the second target image, computing a distance between the lower annulus of the second target image and the distal end detected in the second target image, and displaying the distance over the second target image.

According to some embodiments of the present invention, there is provided a computer implemented method of generating an image for guiding a trans-catheter aortic valve replacement intervention in a subject, comprising: obtaining at least one non-contrast fluoroscopic image depicting at least an aortic root of a subject, the at least one non-contrast fluoroscopic image excludes injected contrast, detecting at least one fiducial object depicted in the at least one non-contrast fluoroscopic image, computing a location of an annulus line indicating an annulus of a native aortic valve relative to the at least one non-contrast fluoroscopic image according to a mapping between the at least one fiducial object and the annulus line, and generating an overlay over the at least non-contrast fluoroscopic image indicating the annulus line according to the location computed according to the mapping.

Optionally, further comprising iterating the obtaining, the detecting, and the computing the location for a plurality of non-contrast fluoroscopic images that are captured sequentially, for dynamically updating the overlay with a real time location of the annulus line.

Optionally, the at least one fiducial object moves synchronously with beats of a heart, and the iterating is performed for dynamically updating the overlay with the real time location of the annulus line of the heart for tracking motion of the annulus line over beats of the heart.

Optionally, further comprising: obtaining at least one contrast fluoroscopic image depicting at least the aortic root of the subject, the at least one contrast fluoroscopic image including injected contrast, detecting the at least one fiducial object depicted in the at least one contrast fluoroscopic image, detecting the annulus line in the at least one contrast fluoroscopic image, and computing the mapping according to a location of the detected at least one fluoroscopic image and a location of the detected annulus line.

Optionally, further comprising: detecting a contour of an aortic annulus in the at least one contrast fluoroscopic image, analyzing the contour for detecting at least two nadirs of at least two cusps of a native aortic valve, and computing the annulus line as a line intersecting the at least two nadirs.

Optionally, further comprising: segmenting pixels denoting contrast from the at least one contrast fluoroscopic image, wherein the contour is detected as a distal border of the segmented pixels.

Optionally, the at least one fiducial object comprises a catheter, and further comprising analyzing the at least one contrast fluoroscopic image for identifying that the catheter is located within at least one cusp of the native aortic valve.

Optionally, the at least one contrast fluoroscopic image is analyzed for identifying that the catheter is located within at least one cusp of the native aortic valve by tracking the location of the detected catheter between a plurality of contrast fluoroscopic images, and verifying that motion of the location of the detected catheter corresponds to expected movement of at least one leaflet of the native aortic valve in response to heartbeats.

Optionally, the contour is detected by segmenting pixels with contrast in proximity to the at least one fiducial object, and identifying the contour of the segmented pixel.

Optionally, further comprising: accessing a 3D image of the subject obtained pre-procedure, and an annulus plane computed for the 3D image, wherein the annulus plane is defined within a 3D space of the 3D image, detecting a change in at least one of a subject orientation, a zoom and a pose of an image sensor capturing fluoroscopic images, projecting the annulus plane defined for the 3D image to a 2D plane corresponding to an orientation of the image sensor according to the detected change, to obtain a new annulus line, and updating the overlay to depict the new annulus line.

Optionally, the annulus plane is computed for the 3D image by: automatically identifying three nadirs of three cusps of an aortic valve; and computing a best fit and/or an intersection of a 2D data structure of the annulus plane defined within the 3D space of the 3D image, to the three nadirs.

Optionally, further comprising registering the annulus line to the annulus plane within the 3D space of the 3D image.

Optionally, further comprising in response to the change, aligning a current fluoroscopic image captured after the change to a preceding fluoroscopic image obtained prior to the change, according to optical flow analysis, for compensating for movement of the subject and/or zoom of the image sensor, and/or for maintaining the registration between the 3D annulus segmented from the 3D image and the annulus line of the fluoroscopic image.

Optionally, the at least one fiducial object includes one or more of: an accessory catheter, a pigtail catheter, at least one synchronized structure that moves in synch with the beating heart, inferior posterior and/or anterior superior regions of the membranous septum floor, and/or the inferior part of the pyramidal space, patterns of calcification signature, tools inserted into the body, a pacing wire, a guidewire, and a stiff guidewire.

According to some embodiments of the present invention, there is provided a system for guiding a medical intervention comprising: at least one processor executing a code for any one of the aforementioned method features.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of components of a system for generating an image for guiding a trans-catheter aortic valve replacement intervention in a subject, in accordance with some embodiments of the present invention;

FIG. 2 is a flowchart of a method for generating an image for guiding a trans-catheter aortic valve replacement intervention in a subject, in accordance with some embodiments of the present invention;

FIG. 3 is a flowchart of a method for training a machine learning model for generating an image for guiding a trans-catheter aortic valve replacement intervention in a subject, in accordance with some embodiments of the present invention;

FIG. 4 is an image that includes a marking indicating a detected valve annulus, and metrics computed based on the detected valve annulus, in accordance with some embodiments of the present invention;

FIG. 5 is another image that includes a marking indicating a detected valve annulus, and metrics computed based on the detected valve annulus, in accordance with some embodiments of the present invention;

FIG. 6 is an image that includes a detected lower plane of the subject aortic valve's cusps and a lower plane of the prosthetic device for computing a device depth, in accordance with some embodiments of the present invention;

FIG. 7 is a flowchart of a method of tracking an anatomical structure of a subject, in accordance with some embodiments of the present invention;

FIG. 8 is an image depicting an annulus line, a fiducial object, and a spatial relationship, in accordance with some embodiments of the present invention;

FIG. 9 is a flowchart of another exemplary method for tracking an anatomical structure of a subject, in accordance with some embodiments of the present invention;

FIG. 10 is a 2D non-contrast fluoroscopic image depicting a calcification signature and a pigtail catheter (i.e., fiducial object), in accordance with some embodiments of the present invention;

FIG. 11 is a 2D contrast enhanced fluoroscopic image depicting an annulus contour, a pigtail catheter (i.e., fiducial object) and a calcification signature, in accordance with some embodiments of the present invention;

FIG. 12 is a flowchart of a method of detecting an annulus on a non-contrast image by correlating with a 3D image, in accordance with some embodiments of the present invention;

FIG. 13 is a flowchart of a process for computing a distance between an annulus line and a distal end of a THV, in accordance with some embodiments of the present invention;

FIG. 14 is a schematic of an exemplary method for tracking an annulus line indicating an annulus of a native aortic valve in fluoroscopic images, in accordance with some embodiments of the present invention;

FIG. 15 includes a schematic 1700A of an exemplary contrast fluoroscopic image 1702 with an overlaid contour 1704 of an annulus of an aortic valve, and a schematic 1700B of exemplary contrast fluoroscopic image 1702 with an overlaid annulus line 1750, in accordance with some embodiments of the present invention;

FIG. 16 is a schematic of an exemplary non-contrast fluoroscopic image 1802 depicting a detected accessory catheter 1804 and an overlay of an annulus line 1806 computed by applying a computed mapping to the detected location of accessory catheter 1804, in accordance with some embodiments of the present invention;

FIG. 17 is a schematic of an exemplary non-contrast fluoroscopic image 1902 depicting a detected synchronized structure, in particular a calcification signature 1904 and an overlay of an annulus line 1906 computed by applying a computed mapping to the detected location of calcification signature 1904, in accordance with some embodiments of the present invention;

FIG. 18 is a schematic of a fluoroscopic image 1802 including an annotation including an overlay of two dashed parallel lines 1804 and 1806, in accordance with some embodiments of the present invention; and

FIG. 19 is a 3D image 2100 of a left ventricle 2102, where an annulus plane 2104 is computed and/or segmented, in accordance with some embodiments of the present invention.

### DETAILED DESCRIPTION

The present invention, in some embodiments thereof, relates to aortic valve replacement and, more specifically, but not exclusively, to systems and methods for guiding aortic valve replacement.

As used herein, the term annulus, which is detected based on images as described herein, may refer to a virtual annulus. The real annulus (of the Aortic valve) is a fibrous ring at the aortic orifice to the front and right of the atrioventricular aortic valve and is considered the transition point between the left ventricle and aortic root. The annulus is part of the fibrous skeleton of the heart. The annulus line and/or virtual annulus detected in images as described herein, refers to the location on the image expected to correspond to the location of the real annulus. The term annulus, lower annulus, annulus contour, contour of the annulus, annulus line, annulus plane, annulus of the heart, annulus of the valve (e.g., aortic valve, tricuspid valve, pulmonary valve) and virtual annulus may be used interchangeably. Alternatively, the contour of the annulus may refer to one or more curves of the annulus, such as a border between the contrast enhanced aortic root and surrounding tissues (without contrast). The annulus line may refer to a line drawn at nadir of cusps of the aortic valve. The annulus line may be computed using a 2D image. The annulus plane may be computed using a 3D image.

As described herein, images may depict injected contrast, or may not depict injected contrast (also referred to herein as excluding injected contrast). The contrast which is depicted or not depicted (excluded) from images may be administered by injection into the body of a subject depicted in the images. Contrast injection may refer to the process where a substance is used to increase the contrast of structures or fluids within the body in medical imaging.

As used herein, the terms fluoroscopy and fluoroscopic are used interchangeably. For example, the terms fluoroscopic image and fluoroscopy image are interchangeable.

As used herein, the terms fiducial object(s) and landmark features may sometimes be interchanged.

As used herein, the term mapping may be an example of a spatial relationship. The term mapping and spatial relationship may sometimes (where relevant) be interchanged. The term mapping may sometimes (where relevant) be interchanged with another example of the spatial relationship, such as vector defining a distance and/or location between two (or more) objects, as described herein. Alternatively, the term mapping may refer to an approach for computing an expected location of a second object relative to a first object (e.g., on a 2D fluoroscopy image, optionally without contrast) using a previously computed spatial relationship between the first object and the second object (e.g., on a pre-procedure CT and/or on a 2D fluoroscopy image with contrast).

It is noted that the terms first anatomical structure and second anatomical structure are used interchangeably, and are not limiting. Rather, what is significant is whether a certain anatomical structure is visible on contrast enhanced images (and not visible or difficult to visualize on non-contrast images), or visible on non-contrast images (and not visible or difficult to visualize on contrast enhanced images). For example, calcification deposits are visible on non-contrast images but are difficult to visualize on contrast enhanced images. Sometimes calcification deposits are referred to as first anatomical structures, and sometimes as second anatomical structures, for example, according to the context of the description and/or in the summary.

As used herein, the term machine learning (ML) model is an exemplary implementation for processing images and/or features extracted from images, for example, a detector, one or more neural networks of various architectures (e.g., convolutional, fully connected, deep, encoder-decoder, recurrent, graph, combination of multiple architectures), support vector machines (SVM), logistic regression, k-nearest neighbor, decision trees, boosting, random forest, a regressor and the like. In some embodiments, other image processing approaches may be used in addition to, and/or alternatively to, the ML model, such as edge detection, feature extraction, masking, and the like.

An aspect of some embodiments of the present invention relates to systems, methods, computing devices, and/or code instructions (stored on a data storage device and executable by one or more processors) for generating an image indicating an annulus line of an annulus of a native aortic valve. The image may be used for guiding a trans-catheter aortic valve replacement intervention in a subject. One or more non-contrast fluoroscopic images that exclude injected contrast are obtained. The non-contrast fluoroscopic images depicting at least an aortic root of a subject, which includes the annulus. The annulus line cannot be seen, and/or cannot accurately be seen, in the non-contrast fluoroscopic images(s). One or more fiducial objects depicted in the non-contrast fluoroscopic image(s) are detected. The fiducial objects may be an accessory catheter (e.g., pigtail catheter used to inject the contrast) and/or other synchronized structures (e.g., calcification signature in the heart) that move in a synchronized manner with the beating heart. A location of the annulus line may be computed relative to the detected location of the fiducial object(s), by applying a previously computed mapping to the location of the fiducial object(s). The mapping is previously computed using a location of the annulus line detected on a contrast fluoroscopic image (i.e., the annulus line may be accurately determined on a fluoroscopic image with injected contrast) and a corresponding location of the fiducial marker(s) detected on the same contrast fluoroscopic image. An overlay overlaid on the non-contrast fluoroscopic image indicating the annulus line, is computed. The overlay may be dynamically updated with a new location of the annulus line for sequentially obtained non-contrast images. The location of the annulus line may change as the heart is beating, and/or in response to displacement of the subject and/or displacement of the image sensor that is capturing the fluoroscopic images.

In some embodiments, one or more anatomical structures of the heart of the patient are detected, optionally the annulus line. Optionally, contrast material may be injected. Image(s) depicting the injected contrast may be analyzed, for example, 2D fluoroscopic images. The images with contrast material may be analyzed to detect the annulus line. The contrast material may be injected via the pigtail catheter. The annulus line may be automatically detected by detecting the contour of the lower boundary of the contrast filled region (e.g., aortic bulb), which may include two or more cusps of the native valve. The nadirs of the two or more cusps may be automatically identified. The annulus line may be defined as the line joining the two or more nadirs. Alternatively, no contrast material is injected. The 2D images exclude contrast. The annulus and/or coronary ostia may be detected by registration of the 2D image that excludes contrast to the 3D image obtained in the pre-processing step. The 3D image of the subject (e.g., CT, MRI) which may be obtained prior to the procedure, which depicts an annulus plane and/or cusps and/or nadir of cusps, may be projected to a 2D plane according to a pose of the image sensor that captured the contrast image. The 2D plane may be registered to the contrast image. The annulus line of the 2D plane may be mapped to the registered contrast image.

At least some embodiments described herein address the technical problem of automatically tracking the annulus of the native aortic valve (i.e., annulus line) on non-contrast fluoroscopic images. At least some embodiments described herein improve the technology of image processing, by automatically tracking the annulus of the native aortic valve (i.e., annulus line) on non-contrast fluoroscopic images. In accordance with the nominal standard TAVR procedure, the operator (e.g., physician) may use fluoroscopy, specifically contrast agent injection in a cine sequence of fluoroscopic images, to visually identify and establish a reference line denoting the annulus line. The annulus line is used to help guide the TAVR procedure, for example, for determining where to deploy the aortic valve prosthesis device. The annulus line is to be recalled by the healthcare provider following the dissipation of contrast material. The act of annotating the annulus line onto a static fluoroscopy image may pose challenges and may result in inaccuracies, since the annulus line cannot be seen or cannot accurately be seen on non-contrast fluoroscopic images.

At least some embodiments described herein address the aforementioned technical problem, and/or improve the aforementioned technical field, by generating an overlay over a non-contrast fluoroscopic image that indicates the location of the annulus line. The location of the annulus line may first be determined on a contrast fluoroscopic image. A location of one or more fiducial markers, optionally an accessory catheter (e.g., pigtail) which may be located in a cusp of the aortic valve, and/or a synchronized structure, may be determined. The fiducial marker(s) may move in a manner that is synchronized with the beating heart. Using the synchronized structure(s) and/or accessory catheter, which moves synchronized to the beating heart, may improve accuracy of computing the location of the annulus, which moves synchronized to the beating heart in a manner corresponding to movement of the fiducial marker(s). A mapping between the location of the annulus line, detected on the contrast fluoroscopic image, and the fiducial marker(s) detected on the contrast fluoroscopic image, is computed. The location of the fiducial marker(s) is then determined on a non-contrast fluoroscopic image. The mapping is applied to the location of the fiducial marker(s), to obtain the location of the annulus line. An overlay that includes the computed location of the annulus line relative to the location of the fiducial marker(s) is generated. The overlay is overlaid on the non-contrast image for indicating the location of the annulus line.

An aspect of some embodiments of the present invention relates to systems, methods, computing devices, and/or code instructions (stored on a data storage device and executable by one or more processors) for generating an image for guiding a trans-catheter aortic valve replacement intervention in a subject. A processor obtains at least one fluoroscopic image depicting an aortic valve prosthesis device in an aorta of the subject. The fluoroscopic image(s) excluding injected contrast, which makes it difficult to visually detect the aortic valve prosthesis and/or anatomical landmarks for correct implantation of the aortic valve prosthesis. The processor feeds the fluoroscopic image(s) into a machine leaning model. The ML model may be trained on a training dataset of fluoroscopic images with injected contrast that depicts the aortic valve prosthesis, and ground truth labels of indications for correct implantation (e.g., markings, anatomical landmarks, and others described herein). The ML model may generate an indication for a correct implantation for implanting the aortic valve prosthesis device in the aorta. In some embodiments, the ML model may generate an indication of the annulus line of the heart of the subject. The indication of the annulus line of the heart of the subject may indicate the physician the implantation location during the procedure. The processor may present the indication as a visual overlay over the at least one fluoroscopic image on a display, for example, color coding the indication in the fluoroscopic image, text/numbers presented on the display, markers on the display (for example: the indication of the annulus line of the heart of the subject may be displayed as a line) and/or audio sounds played over speakers. The ML model may be fed into real time for real time tracking of the indication, to provide real time guidance to the operator for more accurate deployment of the aortic valve prosthesis.

An aspect of some embodiments of the present invention relates to systems, methods, computing devices, and/or code instructions (stored on a data storage device and executable by one or more processors) for training a machine learning model for guiding a trans-catheter aortic valve replacement intervention in a subject. A processor creates a training dataset that includes multiple records. A record includes at least one of: at least one fluoroscopic image depicting an aortic valve prosthesis device in an aorta of the subject that excludes injected contrast, and the at least one fluoroscopic image that includes injected contrast. The record may further include a ground truth label of an indication for a correct implantation for implanting the aortic valve prosthesis device in the aorta. The record may further include a ground truth label of an indication of the indication for the annulus line of the heart of the subject. The training dataset may include records with images of the subject acquired prior to the procedure that exclude the aortic valve prosthesis, for example, slices from a CT scan used for pre-procedure planning and/or MRI scan. The processor trains the ML model on the training dataset.

An aspect of some embodiments of the present invention relates to systems, methods, computing devices, and/or code instructions (stored on a data storage device and executable by one or more processors) for generating an image for guiding a medical intervention (e.g., a trans-catheter aortic valve replacement intervention) in a subject. A processor obtains at least one fluoroscopic image. The processor may automatically track a first anatomical structure (e.g., calcification signature) in fluoroscopic images taken during the intervention, e.g., when no contrast was injected, and use such first anatomical structure for co-locating a second anatomical structure (e.g., annulus line). The processor may further present such second anatomical structure on a display (e.g., as an overlay on the images). The second anatomical structure may be displayed on the display continuously during the intervention.

In some embodiments, a spatial relationship between the first anatomical structure (e.g., calcification signature of the specific patient) and the second anatomical structure (e.g., the annulus line of the patient) may be used for co-locating the second anatomical structure.

The spatial relationship between the first and second anatomical structures may be calculated from fluoroscopic image of the subject taken when the subject was injected with contrast during the intervention. Additionally or alternatively, the spatial relationship between the first and second anatomical structures may be calculated from CT or MRI image of the subject taken prior to the intervention.

In some embodiments, during the intervention, the spatial relationship between the two structures may be detected by image processing methods, for example: by cross correlation the image (taken when no contrast was injected) with template of calcium signature of the subject. Once the calcium signature of the subject was identified in the image (e.g., its position), the annulus line of the subject may be calculated based on the spatial relationship identified.

In some embodiments, during the intervention, the spatial relationship between the two structures may be detected by machine learning methods.

At least some embodiments described herein address the technical problem of guiding a trans-catheter aortic valve replacement procedure. Such procedures are commonly performed under fluoroscopic imaging, which makes it difficult to visualize features of the prosthetic valve that is being deployed and/or anatomical features of the aorta and/or heart in which the prosthetic valve is being deployed. Administration of contrast enhances the visualization, but the amount of contrast that can be administered is physiologically limited, since too much contrast can harm the body of the subject. At least some embodiments described herein improve the medical technology of guiding a trans-catheter aortic valve replacement and/or improve the technology of machine learning.

While procedures for transcatheter implantation of an aortic valve procedure are substantially less invasive than open heart surgery, the lack of line-of-sight visualization of the prosthetic heart valve and the native valve presents challenges, because the physician cannot see the actual orientation of the prosthetic heart valve during the implantation procedure. Correct positioning of the prosthetic heart valve is achieved using radiographic imaging, which yields a two-dimensional image of the viewed area. The physician must interpret the image correctly in order to properly place the prosthetic heart valve in the desired position. Failure to properly position the prosthetic heart valve sometimes leads to migration of the prosthetic heart valve or to improper functioning. Proper placement of the prosthetic heart valve using radiographic imaging is thus critical to the success of the implantation.

At least some embodiments described herein address the aforementioned technical problem, and/or improve the aforementioned technical field, by training a ML model on fluoroscopic images with contrast in which indications for a correct implantation (e.g., anatomical landmarks and/or physical features of the prosthetic valve, the annulus line of the heart of the subject and/or others as described herein) are labelled as ground truth. The training images may exclude the prosthetic valves. The ML model learns to detect the indications on the non-contrast enhanced images based on the training on contrast enhanced images. The images fed into the ML model may be corrected based on images that exclude the prosthetic valve and/or with contrast, for example, CT images obtained pre-procedure.

An aspect of some embodiments of the present invention relates to systems, methods, computing devices, and/or code instructions for automatically detecting an annulus within a native valve (e.g., aortic valve). The annulus may be detected on a contrast image that depicts injected contrast (e.g., into the aortic root). The annulus may be detected on the contrast image, by analyzing the image to detect a respective nadir of two or more cusps of the valve. The annulus may be defined as a line through the two or more nadir. A spatial relationship (e.g., distance, vector, mapping) may be computed between the annulus and a detected fiducial object in the contrast image, for example, a catheter, such as a pigtail catheter used to introduce the contrast. The annulus may be detected on subsequent target image(s) that exclude contrast, by detecting the fiducial object, and applying the spatial relationship. The detected annulus may be presented, for example, as an overlay over the target image(s) that exclude contrast. The annulus is difficult to directly visualize or cannot be directly visualized on images that exclude contrast. At least some embodiments described herein address the technical problem of detecting the annulus of a native valve (e.g., aortic valve) on images that exclude contrast. At least some embodiments described herein improve the technical field of image processing by detecting the annulus of the native valve (e.g., aortic valve) on images that exclude contrast.

Determining the location of the annulus is clinically significant, for example, for determining the site to deploy a valve prosthesis device. Using standard approaches, the location of the annulus may be estimated by repeated contrast injections. It is clinically desirable to reduce injection of contrast into the blood of a subject, since for example, contrast places extra stress on the kidneys, there is a maximum amount of contrast that can be injected, and excessive contrast may be associated with other risks such as allergic reaction.

At least some embodiments described herein provide a technical solution to the aforementioned technical problem, and/or improve the aforementioned technical field, and/or improve upon the aforementioned prior approaches, by enabling detecting and/or tracking of the annulus on images that exclude contrast, for example, presenting the location of the annulus as an overlay. The location of the annulus may be determined based on a spatial relationship between the annulus (which is first determined by analyzing at least one image with contrast) and a fiducial object (e.g., catheter), as described herein.

An aspect of some embodiments of the present invention relates to systems, methods, computing devices, and/or code instructions, for detecting an annulus line on non-contrast images, for example, 2D fluoroscopic images of a native aortic valve. A 3D image depicting the native valve may be obtained, for example, during a pre-procedure imaging session (e.g., CT, MRI). The annulus plane may be detected, for example, as the plane intersecting three nadirs of cusps of the aortic valve. A pattern of calcification deposits, i.e. calcification signature, within the native valve and/or in tissues in proximity to the native valve may be detected. During the procedure, a 2D image may be registered to the 3D image. The 2D image depicts the pattern of calcification, i.e. calcification signature, at a certain pose according to the pose of the image sensor. The registration may be performed by computing the projection plane of the 3D image to a 2D plane that substantially matches the 2D image, according to the pose of the image sensor (e.g., obtained from a sensor installed on the image sensor and/or optical character recognition of text of the image indicating the pose) and by correlating the pattern of calcification deposits, i.e. calcification signature. The annulus plane of the 3D image can be mapped to the 2D image by applying the computed projection plane.

At least some embodiments described herein address the technical problem of automatically determining an annulus line on 2D images that exclude contrast, and/or improves the technology of image processing by automatically determining an annulus line on 2D images that exclude contrast. It is clinically desirable to reduce injection of contrast into the blood of a subject, since for example, contrast places extra stress on the kidneys, there is a maximum amount of contrast that can be injected, and excessive contrast may be associated with other risks such as allergic reaction. At least some embodiments described herein solve the aforementioned technical problem, and/or improve the aforementioned technology, by computing the projection plane of a pre-procedure 3D image to a 2D plane that substantially matches the 2D image, according to the pose of the image sensor (that captures the 2D image) and by correlating a pattern of calcification deposits, i.e. calcification signature. The annulus plane of the 3D image can be mapped to the 2D image by applying the computed projection plane.

An aspect of some embodiments of the present invention relates to systems, methods, computing devices, and/or code instructions for tracking a first anatomical structure (e.g., annulus line) on a non-contrast image. The first anatomical structure (e.g., annulus line) is visible on contrast enhanced images (e.g., where the contrast is injected into the aortic root) but not visible or very difficult to visual on the non-contrast image. The first anatomical structure (e.g., annulus line) may be detected based on a previously defined co-location between the first anatomical structure (e.g., annulus line) and a second anatomical structure visible on non-contrast images (e.g., calcification signature), optionally using a fiducial object depicted in the contrast images and the non-contrast images. The first anatomical structure may be detected based on a combined mapping that is a combination of a first sub-mapping and a second sub-mapping. The first sub-mapping is between the first anatomical structure and the fiducial object. The second sub-mapping is between the second anatomical structure and the fiducial object. Using the combined mapping, the first anatomical structure may be detected on a non-contrast image, according to the detected second anatomical structure, without requiring the fiducial object. The first anatomical structure may be tracked on subsequent non-contrast images.

An aspect of some embodiments of the present invention relates to systems, methods, computing devices, and/or code instructions for tracking an anatomical structure (e.g., annulus line) on a non-contrast image. A 2D target image depicting a native valve of a subject is obtained, for example, a fluoroscopic image during a TAVI procedure. A calcification signature is detected in the 2D target image, for example, a pattern of calcification deposits in the tissues in proximity to the native valve. A calcification template is selected from multiple calcification templates of a 3D image (e.g., CT, MRI, obtained pre-procedure) of the subject according to a pose of the image sensor that captured the 2D target image. The structure (e.g., an annulus plane) is indicated on the 3D image. The 3D image may be projected to a 2D plane according to the pose of the image sensor that captured the 2D target image to obtain the calcification template. The annulus plane of the 3D is mapped to an annulus line on the calcification template. The 2D target image is registered to the calcification template. The annulus line on the calcification template is mapped to the registered 2D target image.

At least some embodiments described herein address the technical problem of automatically determining an annulus line on 2D images that exclude contrast, and/or improves the technology of image processing by automatically determining an annulus line on 2D images that exclude contrast. It is clinically desirable to reduce injection of contrast into the blood of a subject, since for example, contrast places extra stress on the kidneys, there is a maximum amount of contrast that can be injected, and excessive contrast may be associated with other risks such as allergic reaction. At least some embodiments described herein solve the aforementioned technical problem, and/or improve the aforementioned technology, by computing the combined mapping as the combination of the first sub-mapping and the second sub-mapping, as described herein. Using the combined mapping, the first anatomical structure may be detected on a non-contrast image, according to the detected second anatomical structure, without requiring the fiducial object. The first anatomical structure may be tracked on subsequent non-contrast images.

At least some embodiments described herein address the technical problem and/or medical problem of reducing contrast media administration into a subject undergoing a trans-catheter intervention, optionally a TAVI. The contrast media is injected in order to improve visualization of anatomical features that are difficult to see without contrast, for example, the annulus of the aortic valve. When the contrast media is injected into the left atrium and/or aortic root, the contrast media is quickly dissipated by the fast flowing blood out of the heart. Using some standard approaches, repeated contrast media injections are required to position and/or deploy the transcatheter heart valve (THV). Alternatively or additionally, using other standard approaches, the THV is implanted in the native aortic valve in relation to an accessory catheter (e.g., pigtail) positioned as a marker within and/or in proximity to the native aortic valve. The accessory catheter used as a marker may be used instead of, and/or in addition to, the contrast injection(s). It is clinically desirable to reduce injection of contrast into the blood of a subject, since for example, contrast places extra stress on the kidneys, there is a maximum amount of contrast that can be injected, and excessive contrast may be associated with other risks such as allergic reaction.

At least some embodiments described herein solve the aforementioned technical problem and/or medical problem, by automatically tracking the annulus line in 2D images (e.g., fluoroscopic images) that exclude contrast. Various approaches are described herein, for example, computing a spatial relationship between a fiducial object and the annulus, using trained machine learning models, mapping between anatomical structures visualized under contrast and when contrast is excluded and the fiducial object, using calcium signatures, by correlating the 2D images to 3D images indicating the annulus, and/or approaches described herein.

An aspect of some embodiments of the present invention relates to systems, methods, computing devices, and/or code instructions for computing a device depth of a THV from one or more images, optionally 2D fluoroscopic images. The image depicts a partially expanded THV positioned within the native aortic valve. The device depth is computed as a distance between an annulus line (also referred to herein as lower annulus) and a distal end of the THV. The device depth is used to position the THV.

At least some embodiments described herein address the technical problem of computing a device depth of a THV from images, such as 2D fluoroscopic images (contrast and/or non-contrast). The device depth is used to position the THV. In particular, the problem is to determine the device depth when the image depicts a partially expanded THV. At least some embodiments described herein improve the technical field of image processing by automatically computing a device depth of a THV from images.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random-access memory (RAM), a read-only memory (ROM), an erasable programmable read-only memory (EPROM or Flash memory), a static random-access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

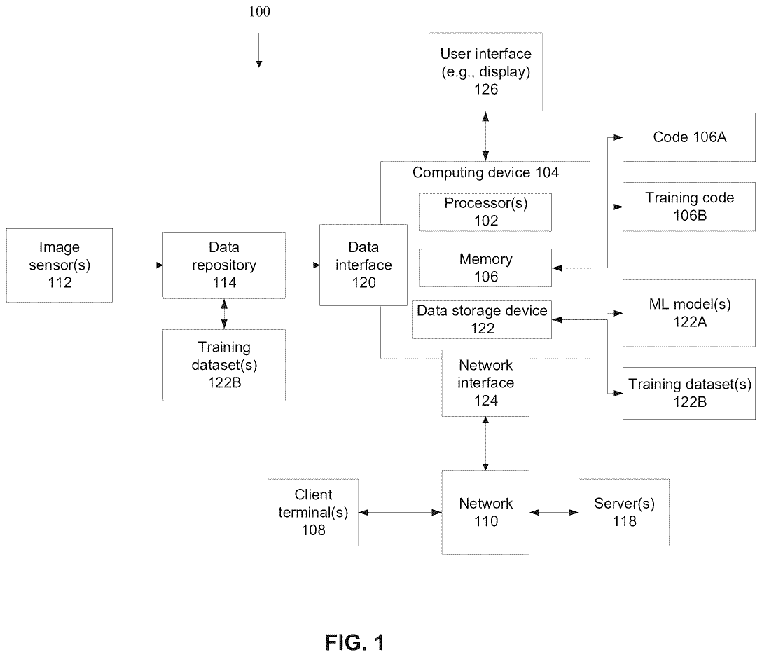

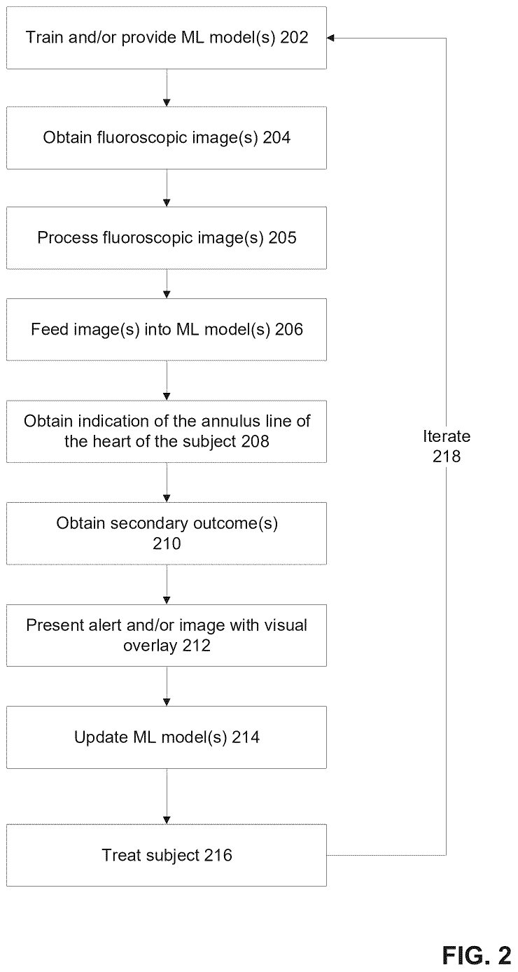

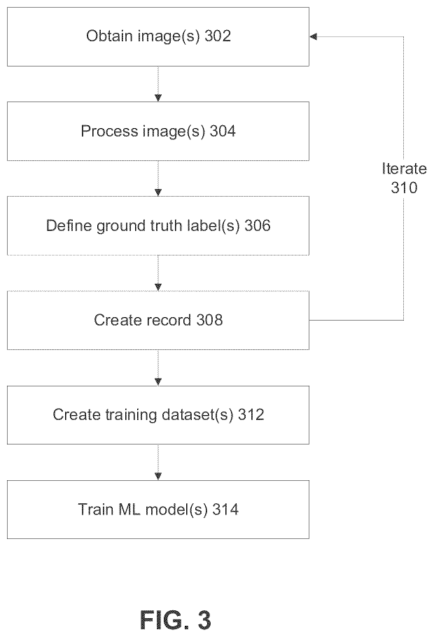

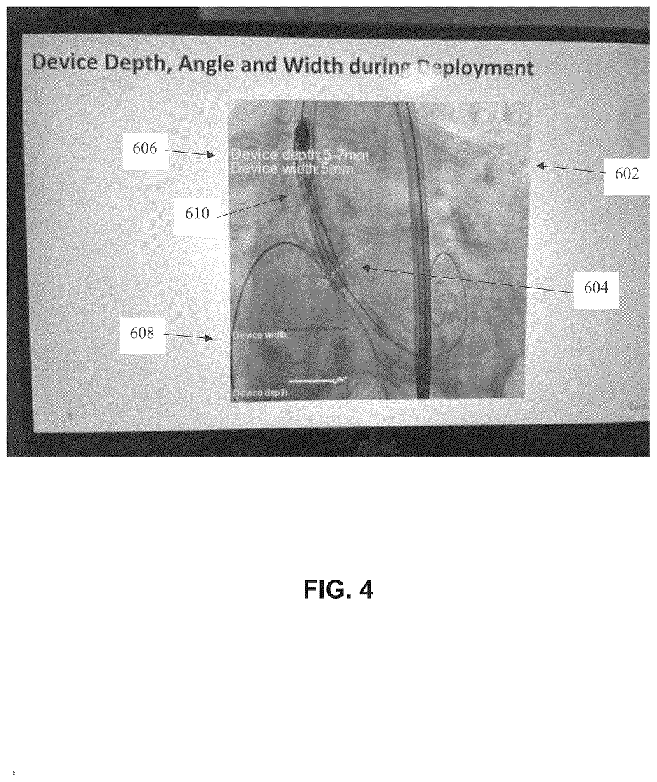

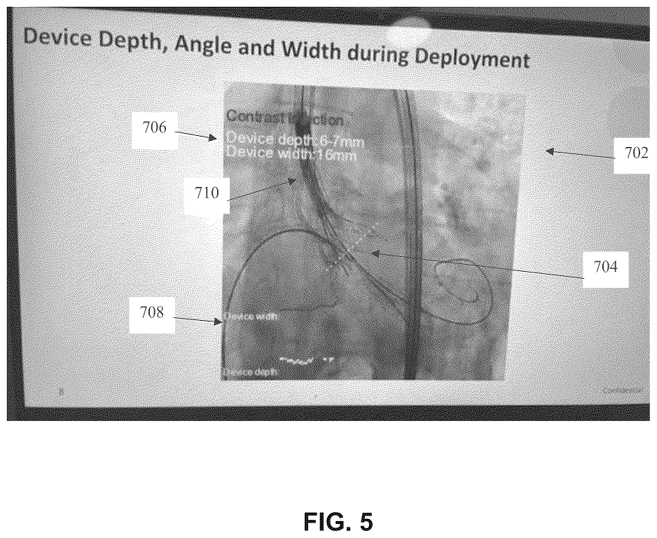

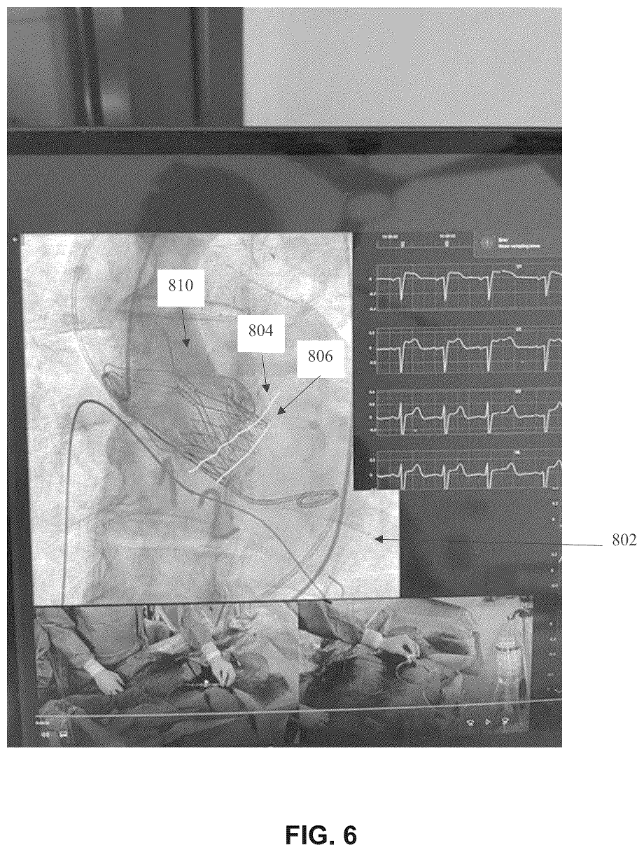

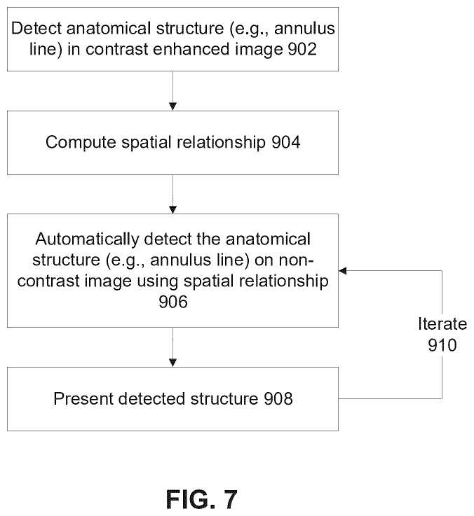

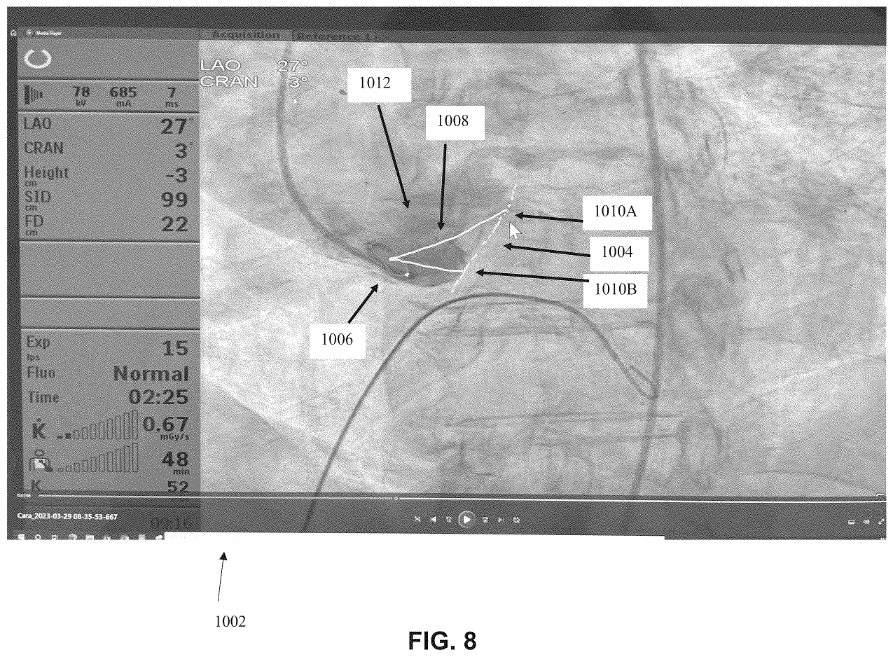

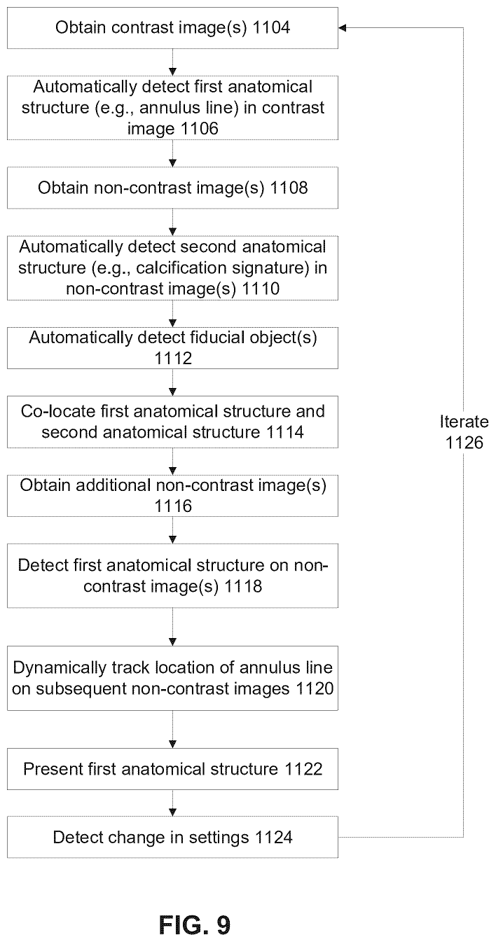

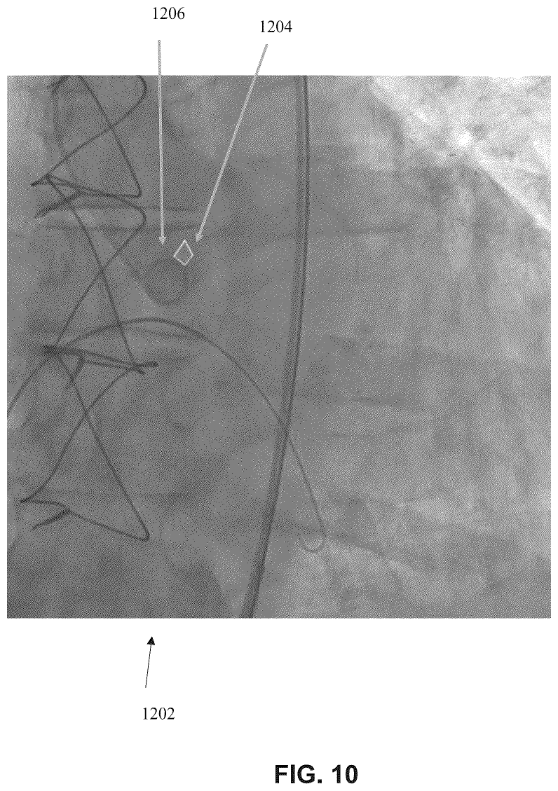

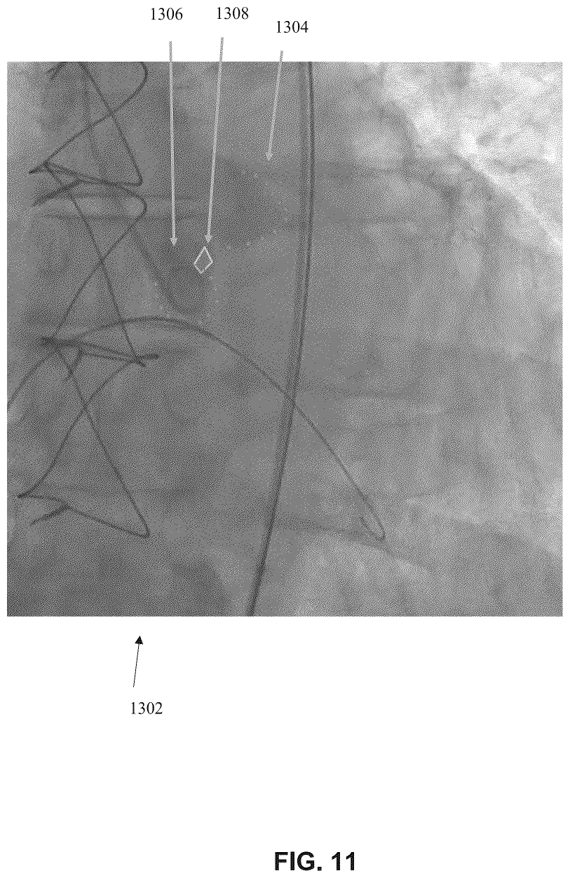

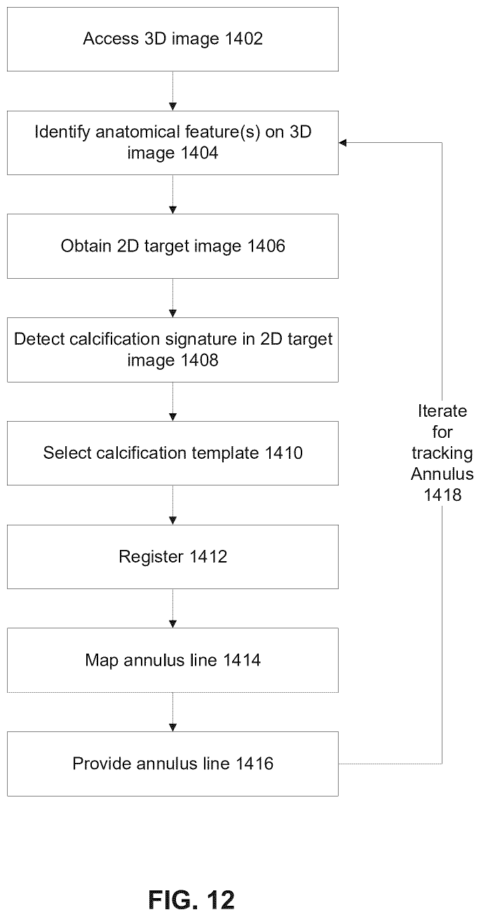

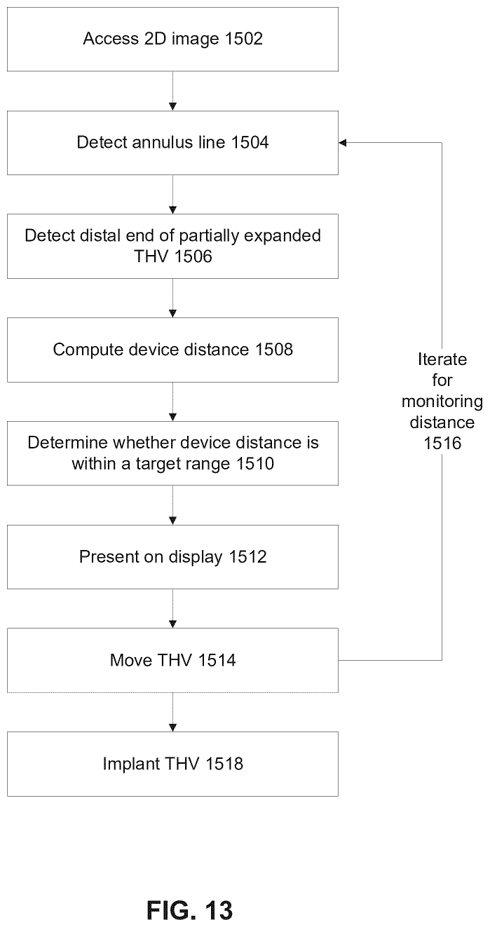

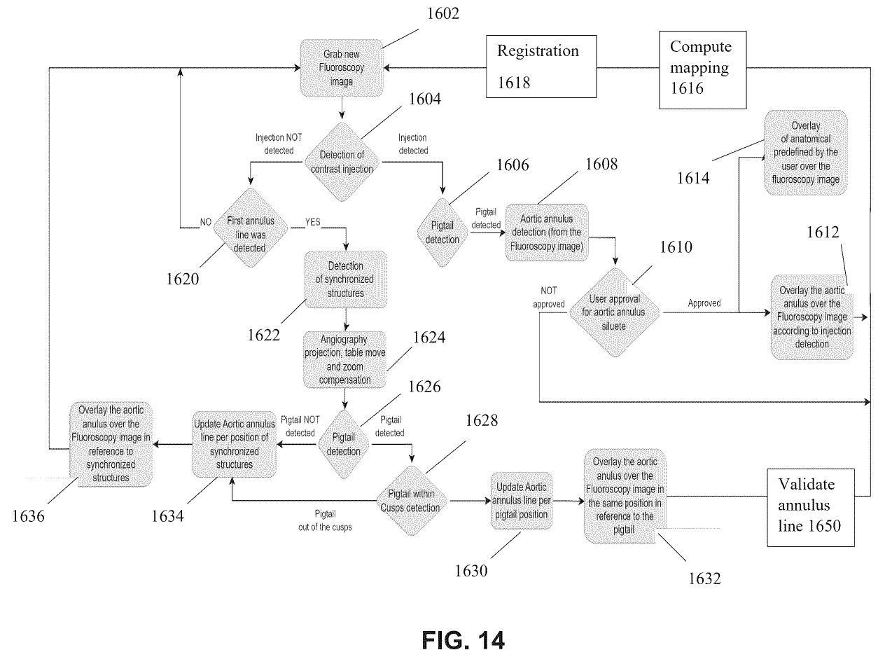

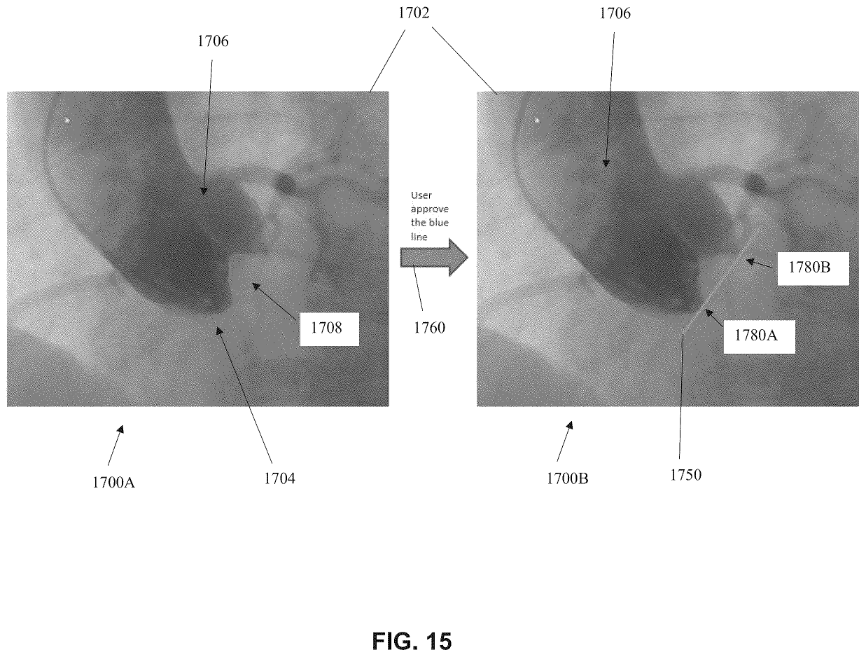

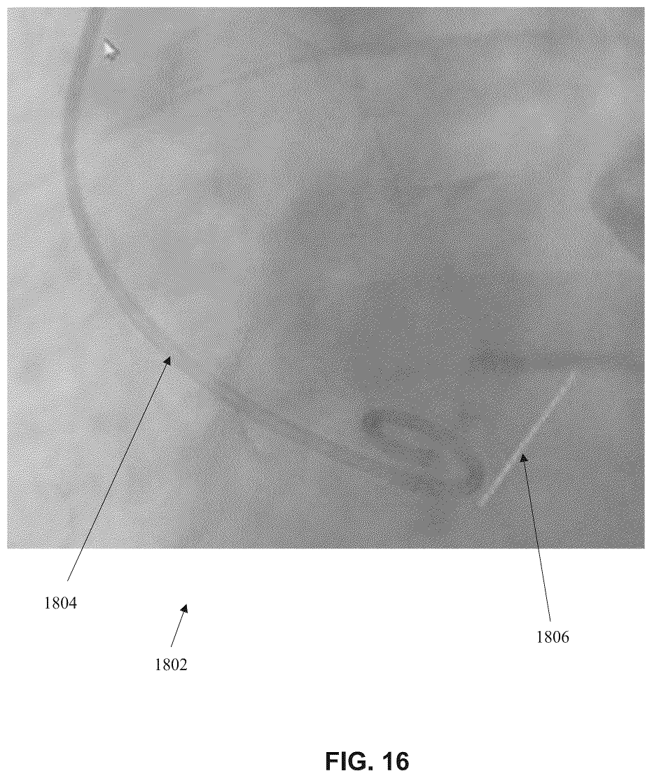

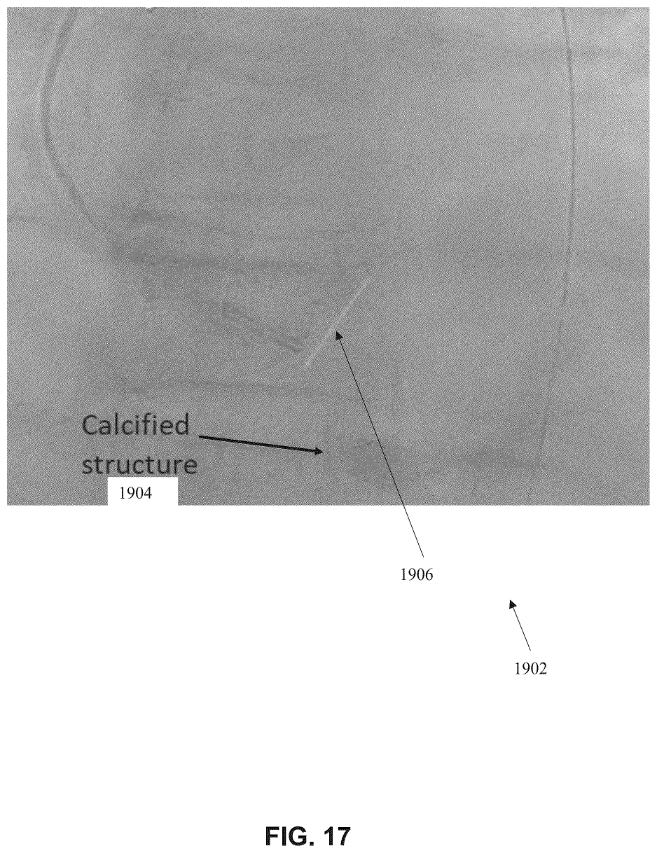

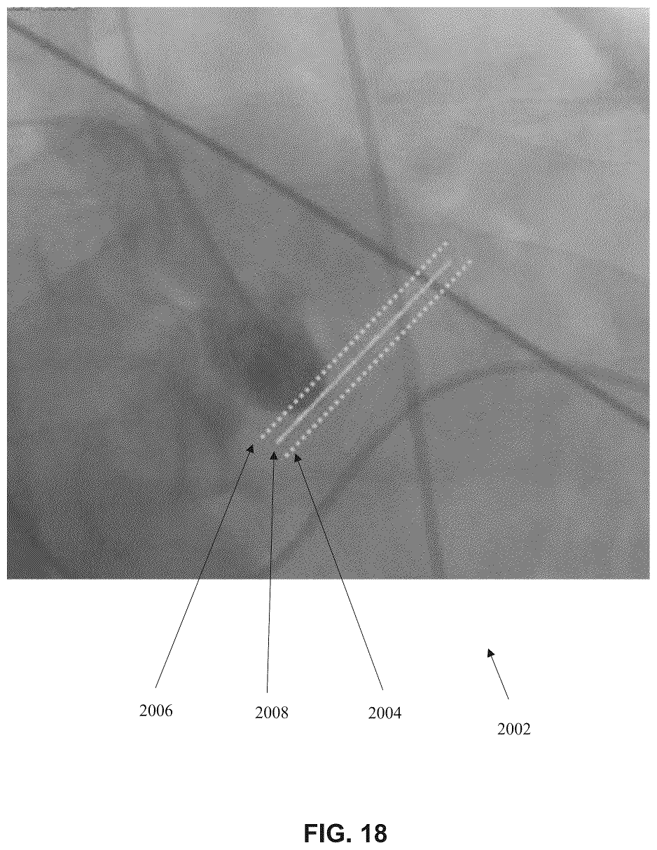

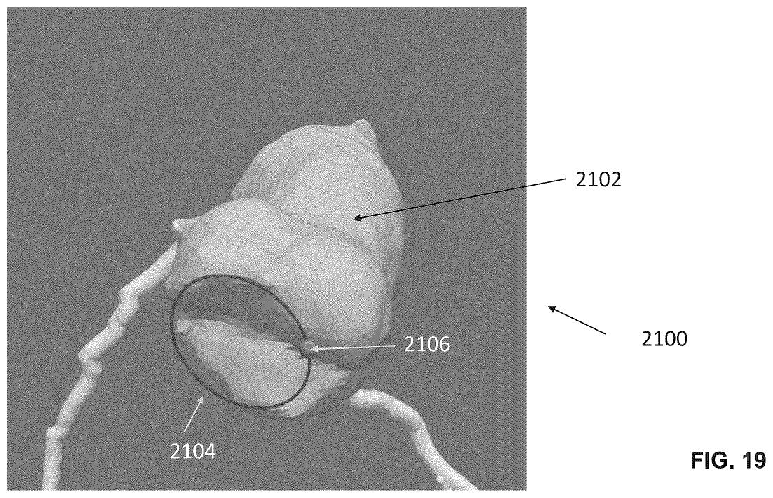

Reference is now made to FIG. 1, which is a block diagram of components of a system 100 for generating an image for guiding a trans-catheter aortic valve replacement intervention in a subject, in accordance with some embodiments of the present invention. Reference is also made to FIG. 2, which is a flowchart of a method for generating an image for guiding a trans-catheter aortic valve replacement intervention in a subject, in accordance with some embodiments of the present invention. Reference is also made to FIG. 3, which is a flowchart of a method for training a machine learning model for generating an image for guiding a trans-catheter aortic valve replacement intervention in a subject, in accordance with some embodiments of the present invention. Reference is also made to FIG. 4, which is an image 602 that includes a marking 604 indicating a detected valve annulus, and metrics 606 608 computed based on the detected valve annulus, in accordance with some embodiments of the present invention. Reference is also made to FIG. 5, which is another image 702 that includes a marking 704 indicating a detected valve annulus, and metrics 706 708 computed based on the detected valve annulus, in accordance with some embodiments of the present invention. Reference is also made to FIG. 6, which is an image 802 that includes a detected lower plane of the subject aortic valve's cusps 804 and a lower plane of the prosthetic device 806 for computing a device depth, in accordance with some embodiments of the present invention. Reference is also made to FIG. 7, which is a flowchart of a method of tracking an anatomical structure of a subject, in accordance with some embodiments of the present invention. Reference is also made to FIG. 8, which is an image 1002 (e.g., fluoroscopic images) depicting an annulus line 1004, a fiducial object 1006, and a spatial relationship 1008, in accordance with some embodiments of the present invention. Reference is also made to FIG. 9, which is a flowchart of another exemplary method for tracking an anatomical structure of a subject, in accordance with some embodiments of the present invention. Reference is also made to FIG. 10, which is a 2D non-contrast fluoroscopic image 1202 depicting a calcification deposit (i.e., calcification signature) 1204 and a pigtail catheter 1206 (i.e., fiducial object), in accordance with some embodiments of the present invention. Reference is also made to FIG. 11, which is a 2D contrast enhanced fluoroscopic image 1302 depicting an annulus contour 1304, a pigtail catheter 1306 (i.e., fiducial object) and calcification deposit (i.e., calcification signature) 1308, in accordance with some embodiments of the present invention. Reference is also made to FIG. 12, which is a flowchart of a method of detecting an annulus on a non-contrast image by correlating with a 3D image, in accordance with some embodiments of the present invention. Reference is also made to FIG. 13, which is a flowchart of a process for computing a distance between an annulus line and a distal end of a THV, in accordance with some embodiments of the present invention. Reference is also made to FIG. 14, which is a schematic of an exemplary method for tracking an annulus line indicating an annulus of a native aortic valve in fluoroscopic images, in accordance with some embodiments of the present invention. Reference is also made to FIG. 15, which includes a schematic 1700A of an exemplary contrast fluoroscopic image 1702 with an overlaid contour 1704 of an annulus of an aortic valve, and a schematic 1700B of exemplary contrast fluoroscopic image 1702 with an overlaid annulus line 1750, in accordance with some embodiments of the present invention. Reference is also made to FIG. 16, which is a schematic of an exemplary non-contrast fluoroscopic image 1802 depicting a detected accessory catheter 1804 and an overlay of an annulus line 1806 computed by applying a computed mapping to the detected location of accessory catheter 1804, in accordance with some embodiments of the present invention. Reference is also made to FIG. 17, which is a schematic of an exemplary non-contrast fluoroscopic image 1902 depicting a detected synchronized structure, in particular a calcification signature 1904 and an overlay of an annulus line 1906 computed by applying a computed mapping to the detected location of calcification signature 1904, in accordance with some embodiments of the present invention. Reference is also made to FIG. 18, which is a schematic of a fluoroscopic image 1802 including an annotation including an overlay of two dashed parallel lines 1804 and 1806, in accordance with some embodiments of the present invention. Reference is also made to FIG. 19, which is a 3D image 2100 of a left ventricle 2102, where an annulus plane 2104 is computed and/or segmented, in accordance with some embodiments of the present invention.

System 100 may implement the acts of the method described with reference to FIGS. 2-19, optionally by a hardware processor(s) 102 of a computing device 104 executing code instructions stored in a memory 106.

Computing device 104 may be implemented as, for example, a client terminal, a server, a virtual server, a radiology workstation, an angiography workstation, a virtual machine, a computing cloud, a mobile device, a desktop computer, a thin client, a Smartphone, a Tablet computer, a laptop computer, a wearable computer, glasses computer, and a watch computer. Computing 104 may include an advanced visualization process that sometimes is add-on to an angiography workstation and/or other devices for presenting overlays on fluoroscopic images for guiding a trans-catheter aortic valve replacement intervention in a subject.

Computing device 104 may include locally stored software that performs one or more of the acts described with reference to FIGS. 2-19, and/or may act as one or more servers (e.g., network server, web server, a computing cloud, virtual server) that provides services (e.g., one or more of the acts described with reference to FIGS. 2-19) to one or more client terminals 108 (e.g., remotely located angiography workstations, remote picture archiving and communication system (PACS) server, remote electronic medical record (EMR) server) over a network 110, for example, providing software as a service (SaaS) to the client terminal(s) 108, providing an application for local download to the client terminal(s) 108, as an add-on to a web browser and/or a medical imaging viewer application, and/or providing functions using a remote access session to the client terminals 108, such as through a web browser.

As used herein, the terms imaging device (e.g., 112) and image sensor are used interchangeably.

Different architectures based on system 100 may be implemented. In one example, computing device 104 provides centralized services. Training of one or more ML models 122A may be performed centrally by computing device 104, as described herein. Computing device 104 may train generic and/or customized ML models. Inference of locally captured fluoroscopic images (e.g., captured by image sensor 112) for detecting indications for guiding deployment of the prosthetic valve may be centrally performed by computing device 104. Alternatively, training is performed by another computing device, and inference is centrally performed by computing device 104. Fluoroscopic images may be provided to computing device 104 for centralized inference by the trained ML model(s) 122A. Images may be provided to computing device 104, for example, via an API, a local application, and/or transmitted using a suitable transmission protocol. The outcome of the inference, including one or more outcomes (e.g., including secondary outcomes) may be provided, for example, to client terminal(s) 108 for presentation on a display and/or local storage, stored in an electronic medical record (e.g., hosted by server 118), and/or stored by computing device 104. In another example, computing device 104 provides centralized training of the ML model(s) 122A, using records to create one or more training datasets 122B provided by different client terminals 108 and/or servers 118. For example, training datasets originating from different hospitals, and/or training dataset for different imaging modalities, and/or for different types of prosthetic valves and/or for different cases such as deployment within a native aortic valve or deployment of another valve within an existing deployed prosthetic valve. Respective generated ML models 122A may be provided to the corresponding remote devices (e.g., client terminal(s) 108 and/or server(s) 118) for local use. For example, each hospital uses the ML model created from their own training dataset for evaluation of new images captured at the respective hospital, and/or different ML models are locally used to evaluate medical images for different types of prosthetic valves.

Image sensor 112 provides the images, which may be included in training dataset(s) 122A and/or for inference. Optionally, image sensor 112 is and/or included in a 2D fluoroscopic and/or x-ray machine, as commonly used in cardiac catheterization labs. Alternatively or additionally, image sensor 112 includes a 2D, 3D, and/or 4D image sensor, such as for capturing pre-procedure images as described herein, for example, a CT machine or a MRI machine. The 3D CT image may be evaluated, and/or 2D slices may be extracted from the 3D CT scan, for example, for training and/or for comparison with 2D real time acquired fluoroscopic images.

Training dataset(s) 122B may be stored in a data repository 114 and/or data storage device 122, for example, a storage server, a computing cloud, virtual memory, and a hard disk. Training dataset(s) 122B are used to train the ML model(s) 122A, as described herein. It is noted that training dataset(s) 122B may be stored by a server 118, accessibly by computing device 104 over network 110.

Computing device 104 may receive images and/or records of the training dataset(s) 122B from image sensor 112 and/or data repository 114 using one or more data interfaces 120, for example, a wire connection (e.g., physical port), a wireless connection (e.g., antenna), a local bus, a port for connection of a data storage device, a network interface card, other physical interface implementations, and/or virtual interfaces (e.g., software interface, virtual private network (VPN) connection, application programming interface (API), software development kit (SDK)).

Hardware processor(s) 102 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include one or more processors (homogenous or heterogeneous), which may be arranged for parallel processing, as clusters and/or as one or more multi core processing units.

Memory 106 (also referred to herein as a program store, and/or data storage device) stores code instruction for execution by hardware processor(s) 102, for example, a random-access memory (RAM), read-only memory (ROM), and/or a storage device, for example, non-volatile memory, magnetic media, semiconductor memory devices, hard drive, removable storage, and optical media (e.g., DVD, CD-ROM). For example, memory 106 may store image processing code 106A that implement one or more acts and/or features of the method described with reference to FIG. 2, and 4-19 and/or training code 106B that execute one or more acts of the method described with reference to FIG. 3.

Computing device 104 may include a data storage device 122 for storing data, for example, one or more trained ML models 122A as described herein and/or one or more training datasets 122B as described herein. Data storage device 122 may be implemented as, for example, a memory, a local hard-drive, a removable storage device, an optical disk, a storage device, and/or as a remote server and/or computing cloud (e.g., accessed over network 110). It is noted that 122A-B may be stored in data storage device 122, with executing portions loaded into memory 106 for execution by processor(s) 102.

Computing device 104 may include a network interface 124 for connecting to network 110, for example, one or more of, a network interface card, a wireless interface to connect to a wireless network, a physical interface for connecting to a cable for network connectivity, a virtual interface implemented in software, network communication software providing higher layers of network connectivity, and/or other implementations. Computing device 104 may access one or more remote servers 118 using network 110, for example, to obtain and/or provide training dataset(s) 116, an updated version of code 106A, training code 106B, and/or the trained ML model(s) 122A.

It is noted that data interface 120 and network interface 124 may exist as two independent interfaces (e.g., two network ports), as two virtual interfaces on a common physical interface (e.g., virtual networks on a common network port), and/or integrated into a single interface (e.g., network interface). Computing device 104 may communicate using network 110 (or another communication channel, such as through a direct link (e.g., cable, wireless) and/or indirect link (e.g., via an intermediary computing device such as a server, and/or via a storage device) with one or more of:
Client terminal(s) 108, for example, when computing device 104 acts as a server providing image analysis services (e.g., SaaS) to remote angiography terminals, for analyzing remotely obtained fluoroscopic images using the trained ML model(s) 122A. Training dataset(s) 122B may be created based on fluoroscopic images received from one or more client terminals 108.
Server 118, for example, implemented in association with a PACS, which may store image for training dataset(s) 122B and/or may store captured fluoroscopic images for inference. Training dataset(s) 122B may be created from fluoroscopic images stored by server 118.
Image sensor 112 and/or data repository 114 that store fluoroscopic images acquired by image sensor 112. The acquired fluoroscopic images may be fed into trained ML model(s) 122A for inference. Training dataset(s) 122B may be created based on fluoroscopic images obtained from one or more image sensors 112.

Computing device 104 and/or client terminal(s) 108 and/or server(s) 118 include and/or are in communication with a user interface(s) 126 that includes a mechanism designed for a user to enter data (e.g., manually define ground truth) and/or view the indication outputted by the ML model(s) such as an overlay over the input image. Exemplary user interfaces 126 include, for example, one or more of, a touchscreen, a display, a keyboard, a mouse, and voice activated software using speakers and microphone.

Referring now back to FIG. 2, at 202, one or more machine learning models are trained and/or trained ML models are accessed. An exemplary approach for training the ML model is described for example with reference to FIG. 3.

In some embodiments, the ML model may be trained for generating an indication for a correct implantation for implanting the aortic valve prosthesis device in the aorta, in response to an input of fluoroscopic images without contrast. In some embodiments, the ML model may be trained for generating an indication of the annulus line of the heart of the subject, in response to an input of fluoroscopic images without contrast.

The correct implantation area (it has several relevant places that might be interesting to know about, in the valve area) for the valve is not visible unless contrast material is injected. The ML model may find places (on the dye-image) and continue marking them on the fluoroscopy image as the procedure progresses.

The ML model decides what landmarks it can use and what it cannot. Pre-operative CT can help with that. In some embodiments, the landmarks include one or more of: anatomical structures around the annulus line and signature of calcification presence in the heart.

The machine learning model may be trained on a training dataset of fluoroscopic image with injected contrast that depicts at least one of the aortic valve prostheses and the indication for the annulus line.

In some embodiments, the ML model may be fine-tuned on the actual calcification signature of the specific patient obtained from pre-procedure CT image of the patient and/or fluoroscopic images taken when the patient was injected with contrast. In some embodiments, the ML model may learn the relation, e.g., spatial relationship, between the annulus line and the calcification signature of the specific patient. During the procedure, the calcification signature of the specific patient may be detected and/or identified from fluoroscopic images taken when no contrast was injected and the annulus line of the patient may be automatically detected based on the spatial relationship between the annulus line and the calcification signature of the specific patient. At 204, the processor obtains one or more fluoroscopic images (also referred to herein as current image(s)) and/or target image, depicting an aortic valve prosthesis device in an aorta of the subject). The fluoroscopic images excluding injected contrast, which makes it difficult or impossible to sufficiently visualize the aortic valve prosthesis and/or the indication for determining a site for implanting the aortic valve prosthesis device and/or the annulus line.

At 205, the processor may pre-process the one or more fluoroscopic images.

Optionally, the processor processes the fluoroscopic image, by correcting its alignment, and feeding the corrected image into the ML model. The processor may process the at least one fluoroscopic image by comparing the fluoroscopic angulation of the at least one fluoroscopic image to at least one image of the training dataset used to train the ML model and/or at least one image acquired before the procedure that excludes the valve prosthesis. The processor may correct a definition of correct alignment use for the at least one fluoroscopic image and/or correct incorrect alignment of the at least one fluoroscopic image.

Alternatively or additionally, the processor extracts landmark features from the fluoroscopic images. The landmark features may be fed into the ML model. For example, the processor identifies multiple landmarks on the fluoroscopic image (e.g., by feeding into another ML model, and/or by image processing code) which are then fed into the ML model to obtain the indication of a native aortic valve annulus. For example, the indication is a detection of an annulus of a native aortic valve, and the landmarks include one or more of: signature of calcification presence in the heart, e.g., in the vicinity of the annulus line, location of commissures of the native aortic valve, entry to at least one coronary artery, and location of a pigtail catheter positioned in one of the coronary cusps of the native aortic valve. The signature of calcification presence in the heart, e.g., in the vicinity of the annulus line, may be calculated from CT or MRI image of the subject, taken pre-procedure. The signature of calcification presence in the heart, e.g., in the vicinity of the annulus line, may be calculated from fluoroscopic image of the subject, taken when contrast was injected. In some embodiment, the ML model may learn the annulus line position in the heart in relation to such signature of calcification presence; as the signature of calcification presence is detected on the image also when no contrast is injected.

Optionally, the processor extracts a location of calcification signature. The processor may feed the location into the ML model for generating an outcome indicating a target anatomical feature, such as location of the aortic plane or annulus line. In other words, the processor may automatically track a first anatomical structure (e.g., calcification signature) in images taken during the procedure, e.g., when no contrast was injected, and used such first anatomical structure for co-locating a second anatomical structure (e.g., annulus line) to present such second anatomical structure on a display (e.g., as an overlay on the images). The second anatomical structure may be displayed on the display continuously during the procedure.

Optionally, the processor extracts two or more landmarks in the image. The two or more landmarks may be fed into the ML model for obtaining the annulus line. Alternatively, the location of the two or more landmarks is not explicitly extracted as a feature, but serves as an implied feature when the two or more landmarks are depicted in the current image.

At 206, the processor may feed the fluoroscopic image into the machine leaning model. Alternatively, the processor feeds the fluoroscopic image into two processing paths. The processor may feed the fluoroscopic image into a first processing path that includes a first ML model that generates a first outcome of an anatomical landmark of the subject, and the processor feeds the same fluoroscopic image into a second processing path that includes a second ML model that generates a second outcome of a physical landmark of the aortic valve prosthesis. The two processing paths enable computing a distance metric as a difference between the first outcome and the second outcome. Optionally, the anatomical landmark of the first outcome is a location of an aortic plane, and the physical landmark of the second outcome is a distal end of the artificial valve prosthesis.

Alternatively, the processor feeds the fluoroscopic image into two processing paths. The processor may feed the fluoroscopic image into a first processing path that includes a first ML model that generates a first outcome of an anatomical landmark of the subject (e.g., calcification signature), and the processor feeds the same fluoroscopic image into a second processing path that includes a second ML model that generates a second outcome of the annulus line of the heart of the subject.

The first ML model may be trained on a first training dataset of records. Each record may include an image in which a region that includes an ascending aorta is masked and a first ground truth label of a location of the anatomical landmark. The second ML model is trained on a second training dataset of records. Each record includes a second complete image with ascending aorta and a second ground truth label of a location of the physical landmark.

At 208, the processor may obtain an indication of the annulus line of the heart of the subject. Optionally, the indication is for a correct implantation for implanting the aortic valve prosthesis device in the aorta.

Exemplary approaches for detecting the annulus line, optionally on non-contrast images, are described herein, for example, with reference to FIG. 7, FIG. 9, FIG. 12 and FIGS. 14-19.

In some embodiments, an ML model is not used and the spatial relationship between the annulus line and the signature of calcification presence of the specific patient and/or the signature of calcification presence of the specific patient may be detected by image processing methods, for example: by cross correlation the image (taken when no contrast was injected) with template of calcification signature of the subject. Once the calcification signature of the subject was identified in the image (e.g., its position), the annulus line of the subject may be calculated based on the spatial relationship identified.

The indication may be an overlay, and/or the input image with overlay. The indication may be text, code, drawing, and the like for generating an alert and/or overlay.

The indication of the annulus line of the heart of the subject is an exemplary implementation. Other structures within a native valve of a subject may be indicated as well.

Optionally, the indication may be at least one anatomical landmark. For example, feature in an anatomical region of the aorta for implantation of the aortic valve prosthesis device. In another example, the anatomical landmark is a location of the entry to the two-coronary artery (e.g., for the purpose of relating the image to the pre procedure CT for indicating beyond commissural alignment of the orientation of the artificial valve with regard to the subject's coronary artery openings, a step termed coronary alignment).

Alternatively or additionally, the indication may be a depth of the artificial valve prosthesis relative to an anatomical landmark of the subject (also referred to herein as device depth). The anatomical landmark may be a lower plane of an annulus (cusps) of an aortic valve of the subject. The depth may be from the anatomical landmark to a lower plane of the artificial valve prosthesis. Optionally, the processor generates an alert indicating correct placement when the depth is between about 3-5 millimeters (mm), or about 2-6 mm, or about 3-4 mm, or about 4-5 mm, or other values. A more precise range may be determined according to a type of the artificial valve, which may be automatically detected (e.g., from the image), from the medical record, and/or manually entered. Optionally, during multiple iterations for multiple fluoroscopic images received in real time, the processor monitors the images to dynamically track the detected device depth (i.e., the distance between the lower plane of the patient aortic valve's cusps, and the lower plane of the implanted device).

Optionally, the indication of the depth is computed by the processor calibrating the depth according to a detected physical feature of the artificial valve prosthesis for which a fixed dimension is known. For example, diamond shaped cells of the artificial valve prosthesis which have a dimension that is known and/or fixed. The calibration is done to convert the device depth from the current image to an actual distance.

Optionally, the ML model generates a first outcome of a location of the artificial valve prosthesis and a second outcome of a location of the anatomical landmark, for example, 3D and/or 2D coordinates, and/or labels for pixels of the current image. The processor computes the depth as a distance between the first outcome and the second outcome, for example, as a Euclidean distance between the two sets of coordinates, and/or between the labelled pixels.

At 210, the processor obtains and/or computes one or more secondary outcomes. It is noted that one or more of the secondary outcomes listed below may be an outcome obtained from the ML model as described with reference to 208. The secondary outcomes may be obtained, for example, from the ML model, from one or more secondary ML models, and/or from an analysis of the images (e.g., by image processing code).

Alternatively or additionally, the secondary outcome is an orientation of a heart in which the valve prosthesis is being implanted. The orientation may be computed by determining an annulus line of the heart by analysing the at least one fluoroscopic image, and computing the orientation relative to the heart. The annulus line may be used to determine if the heart is horizontal.

Alternatively or additionally, the secondary outcome is an opening percentage of the aortic valve prosthesis device. When the opening percentage is below a threshold (e.g., 80%), the operator may revert back, retract and close the prosthetic valve, and reposition the prosthetic valve. The opening percentage may be computed by analysing the aortic valve prosthesis in the at least one fluoroscopic image (e.g., measuring diameter and/or feeding into a secondary ML model), and/or by comparing a diameter of the aortic valve prosthesis to previously obtained fluoroscopic images to determine an amount of increase in the diameter.

Alternatively or additionally, the secondary outcome is the annulus line of the heart. The annulus line for the at least one fluoroscopic image may be computed from two or more previously acquired images at different projections, for example, by computing a transformation from the previously acquired images to the current image, and computing the annulus line based on the transformation. For example, the processor uses the pre planning CT (or other previously acquired image) and current real time fluoroscopy image, and the location of the annulus line at a known projection. These inputs are used for extracting the 3D transformation between subject's pre planning CT anatomy and the real time position. Using this transformation, the processor may draw the annulus line in different fluoroscopy projections, for example, by feeding the new fluoroscopic projection angulation into to a transformer.

Alternatively or additionally, the secondary outcome is the end location of the aortic valve prosthesis device relative to a location of a native valve of the subject. The end location and/or distance relative to the native valve may be computed by analyzing the image(s), by image processing code and/or a secondary ML model, to detect the end of the prosthetic valve and location of the native valve, and computing the difference therebetween. Optionally, maximal distance and minimal distance of the end location are computed by analyzing historical values of the distance of the end locations determined for preceding images.

Alternatively or additionally, the secondary outcome includes one or more of a proximal location of the aortic valve prosthesis device relative to the native valve location, and/or the proximal location of the aortic valve prosthesis device relative to the location of the coronaries'orifices.

Alternatively or additionally, the secondary outcome includes an angle of the aortic valve prosthesis device relative to the native valve orientation.

Alternatively or additionally, the secondary outcome includes a distal end width of the aortic valve prosthesis device.

Alternatively or additionally, the secondary outcome includes a maximal width of the aortic valve prosthesis device, for example, based on historical values of preceding widths computed from preceding images.

Alternatively or additionally, the secondary outcome includes a maximal length of the aortic valve prosthesis device, for example, based on historical values of preceding lengths computed from preceding images.

Alternatively or additionally, the secondary outcome includes an automatically detected change in projection from a previously captured image that excludes the artificial valve prosthesis (e.g., prior obtained CT scan of the subject before the procedure) to the at least one fluoroscopic image for one or more predefined projections, for example, a cusp overlap view, and/or an all cusps view. The projection angle may be computed, and/or the projection label may be presented.

Alternatively or additionally, the secondary outcome includes a depth change of the artificial valve prosthesis while the artificial valve prosthesis is partially or fully opened, and while the artificial valve prosthesis is being dragged downwards towards the heart and/or upwards away from the heart.

Alternatively or additionally, the secondary outcome includes an indication of whether a location of the balloon is too deep or too high.

At 212, the processor may present the indication and/or secondary indication as a visual overlay over the at least one fluoroscopic image on a display. Alternatively or additionally, the indication and/or secondary indication may be an adaptation of the at least one fluoroscopic image. Alternatively or additionally, the indication and/or secondary indication may be an alert, for example, an audio message played on speakers, a text message, a pop-up box, an arrow, and the like, which may be presented on the fluoroscopic image(s) and/or on another display.

At 214, a new record may be formed from the fluoroscopic image(s) and/or the outcomes generated by the ML model(s) and/or the secondary outcome(s). The new record may be used for dynamically updating the ML model(s), for example, the ML model(s) is a generic model trained on records of multiple different subjects and/or trained on a generic model (e.g., rendered as an aggregation of multiple images, a non-human simulation, and the like). The generic model may include a generic artificial aortic valve prosthesis. The new record is a form of transfer learning for customizing the generic ML model for the subject. For example, the ML model may be trained a-priori on the generic model to learn the shapes of the aortic valve's cusps and/or the shape of the artificial aortic valve prosthesis and/or a shape of calcification signature in the heart. During the procedure, the generic ML model may be fine-tuned and/or a transfer learning approach may be used, on the actual aorta shape of the specific patient or the calcification signature of the specific patient, by dynamic training of ML model using training records created from images taken from the specific patient (e.g., in several breathing cycles of the patient, e.g., 1-3 cycles) of the chest fluoroscopy including the area of aortic valve.

In some embodiments, the generic ML model may be fine-tuned and/or a transfer learning approach may be used, on the actual calcification signature of the specific patient obtained from pre-procedure CT image of the patient and/or fluoroscopic images taken when the patient was injected with contrast.

In some embodiments, the ML model may learn the relation, e.g., spatial relationship between the annulus line and the calcification signature of the specific patient.

Optionally, the new record includes a fluoroscopic image of the subject in which contrast has been injected, optionally labelled with one or more of the indications as ground truth. The ML model may be dynamically updated with the new record, which improves inference performance of the ML model on additional fluoroscopic images of the subject without contrast. The ML model may learns the specific anatomy of the subject (e.g., the calcification signature of the specific patient) from the contrast enhanced images to apply to non-contrast enhanced images.

Optionally, the ML model is a generic ML model. The processor dynamically updates the generic ML model to create a customized ML model for the subject by dynamically creating customized records that include fluoroscopic images of the subject, and dynamically training the generic ML model on the customized records. The customized records may include a first set of fluoroscopic images of the subject with contrast and a second set of fluoroscopic images without contrast. Ground truth labels are applied to the customized records that include the first set of fluoroscopic images with contrast, to mark a target anatomical feature (e.g., the calcification signature of the specific patient). The processor generates a mask for the first set of fluoroscopic images with contrast to mask pixels of a target anatomical features, for example, mask a region(s) depicting the aorta and aortic valve. The processor replaces the masked region with spatially corresponding pixels of the region from the second set of fluoroscopic images without contrast to generate a third set of synthesized images. The processor assigns the ground truth labels of the first set to the third set of synthesized images, for creating a third set of records that include the third set with ground truth label. The processor trains the ML model on the third set for detecting the target anatomical feature, i.e., the indication (e.g., aortic plane line) on images without contrast.

In other words, the ML model may “learn” to associate the label of the annulus line of the subject.

Alternatively or additionally to the masking process, the target anatomical feature may be redrawn (e.g., by image processing code) over a plurality of respiratory and/or cardiac cycles with matching frames of the second set of fluoroscopic images without contrast to create a fourth set of training records. The ML model may be self-trained to use the non-contrast images of the fourth set for detecting the target anatomical feature (e.g., aortic plane line).

Optionally, images are processed to exclude pixels of a region of the ascending aorta and optionally a “halo” around the region, for example, 2-6 mm, or about 5 mm around the region.

The ML model may use the learning of the labelling of the aortic plane (and/or aortic frame) independently of the angiogram.

At 216, the subject may be treated based on the alerts and/or visual indications, for example, by deploying the prosthetic aortic valve in the identified correct location such as within the target depth, moving the valve to a different position, retracting the valve, adjusting the guidewire, and the like. In some embodiment, the physician may implant the prosthetic aortic valve based on the indication of the annulus line.

At 218, one or more features described with reference to 202-216 are dynamically iterated during the procedure, for guiding the valve prosthesis to the correct location for deployment and/or for assisting the physician in guiding the valve prosthesis to the correct location for deployment.

For example, the processor may identify and/or alert the operator when a target device depth (e.g., within a target range) is obtained, and/or a recommendation to the user where to deploy the valve prosthesis may be generated. Alternatively or additionally, prior to implantation of the valve prosthesis, the process may detect the annulus line and/or direct the marker of the artificial valve prosthesis to be at the same level.

The features are iterated while the valve is transported via the descending aorta and/or ascending aorta on its way to be implanted in the native aortic annulus, for assisting the operating with real time navigation of the valve.

Referring now back to FIG. 3, it is noted that some features of the training are described with reference to FIG. 2. Those features may also fit into features described with reference to FIG. 3, but are omitted for clarity of reading, to avoid duplication.

At 302, one or more fluoroscopic images are obtained. The fluoroscopic image(s) depict an aortic valve prosthesis device in an aorta of the subject. The image may exclude injected contrast and/or may include injected contrast. At least some images include injected contrast.

Optionally, images of the subject acquired prior to the procedure that exclude the aortic valve prosthesis are obtained, for example, from 2D x-rays and/or a CT scan of the subject, such as done for pre-procedure planning.

At 304, the image may be processed. Processing may be done as described with reference to 205 of FIG. 2, for example, extracting one or more anatomical landmarks or structures. For example, template of calcification signature of the subject, tissue thickening and/or blood flow derived dynamic changes.

At 306, one or more ground truth labels are defined. Ground truth labels may be defined, for example, manually by a user (e.g., placing labels and/or markings on the image via a graphical user interface), automatically by code (e.g., image processing code that automatically identifies certain features), and/or by correlating with another image, for example, labels on a contrast enhanced image are mapped to corresponding locations on a non-contrast enhanced image, where the contrast enhanced image and the non-contrast enhanced image are correlated to each other such as via common key points.

Optionally, the ground truth label is an indication of the annulus line of the heart of the subject, middle of a subject's native aortic valve, top of a subject's native aortic valve, one or more commissures of a subject's native aortic valve, the leaflets of a subject's native aortic valve and the sinuses next to a subject's native aortic valve.

Optionally, such indication may guide the physician for a correct implantation for implanting the aortic valve prosthesis device in the aorta.

At 308, a training record is created. The training record includes the image(s), optionally processed such as the correct image and/or the features extracted from the image, and may include the ground truth labels when available.

In some embodiment, a training record may include a spatial relationship between a first anatomical structure (for example: template of calcification signature of the subject, tissue thickening and/or blood flow derived dynamic changes) and a second anatomical structure (for example: the annulus line of the heart of the subject, middle of a subject's native aortic valve, top of a subject's native aortic valve, one or more commissures of a subject's native aortic valve, the leaflets of a subject's native aortic valve and the sinuses next to a subject's native aortic valve).

The spatial relationship between the first and second anatomical structures may be calculated from fluoroscopic image of the subject taken when the subject was injected with contrast during the intervention.

Additionally or alternatively, the spatial relationship between the first and second anatomical structures may be calculated from CT or MRI image of the subject taken prior to the intervention.

At 310, features described with reference to 302-308 are iterated for creating multiple records.

At 312, one or more training datasets of multiple training records are created.

At 314, one or more ML models are trained on the training dataset(s).

Referring now back to FIG. 4, fluoroscopic image 602 depicts an artificial prosthetic valve 610 for implantation. Line 604, which is automatically generated as an outcome of the ML model in response to an input of image 602, is generated as an overlay of image 602. Line 604 indicates a location of the annulus (i.e., lower plane of the subject's aortic valve cusps). Metrics 606, for example, device depth and device width, are computed based on indications outputted by the ML model, as described herein. Metrics 608 may be, for example, a plot along time indicating variations in values of the width of the device (i.e., the artificial prosthetic valve) as the operator expands and/or retracts the artificial prosthetic valve, and/or depth of the device (i.e., the artificial prosthetic valve) as the operator moves the artificial prosthetic valve proximally and/or distally.

Referring now back to FIG. 5, fluoroscopic image 702 depicts an artificial prosthetic valve 710 for implantation, during contrast injection, and in a more expanded state than valve 610 of FIG. 4. Line 704, which is automatically generated as an outcome of the ML model in response to an input of image 702, is generated as an overlay of image 702. Line 704 indicates a location of the annulus (i.e., lower plane of the subject's aortic valve cusps). Metrics 706, for example, device depth and device width, are computed based on indications outputted by the ML model, as described herein. Metrics 708 may be, for example, a plot along time indicating variations in values of the width of the device (i.e., the artificial prosthetic valve) as the operator expands and/or retracts the artificial prosthetic valve, and/or depth of the device (i.e., the artificial prosthetic valve) as the operator moves the artificial prosthetic valve proximally and/or distally.

Referring now back to FIG. 6, fluoroscopic image 802 depicts an artificial prosthetic valve 810 for implantation. Detected lower plane of the subject aortic valve's cusps 804 and detected lower plane of the prosthetic device 806 are shown. Features 804 and/or 806 may be computed by the ML model, optionally two ML models using the dual path processing, as described herein. The device depth is computed as the difference in distance between features 804 and 806, as described herein.

Reference is also made to FIG. 7, which is a flowchart of a method of tracking an anatomical structure of a subject, in accordance with some embodiments of the present invention. The method enables tracking the anatomical structures using images without injected contrast.

Features described with reference to FIG. 7 may be implemented with, replaced by, combined with, and/or used in addition to, other features of other methods described herein.

At 902, a contrast image (also referred to as a contrast enhanced image) that includes injected contrast, is analyzed to detect the anatomical structure. The anatomical structure may be an annulus of a native valve, optionally an aortic valve. The contrast image may depict the aortic root and surrounding tissues. The contrast may be injected within the aortic root, depicting the contour of the annulus. For example, the contour of the annulus may be the boundary between the bottom of the contrast filled aorta and the surrounding tissues and/or left ventricle without contrast.

The annulus may be automatically detected by detecting the contour of the annulus, which may include two or more cusps of the native valve. The nadirs of the two or more cusps may be automatically identified. The annulus, also referred to as the annulus line, may be defined as the line joining the two or more nadirs.

Alternatively or additionally, a 3D image of the subject (e.g., CT, MRI) which may be obtained prior to the procedure, which depicts an annulus plane and/or cusps and/or nadir of cusps, may be projected to a 2D plane according to a pose of the image sensor that captured the contrast image. The 2D plane may be registered to the contrast image. The annulus line of the 2D plane may be mapped to the registered contrast image.

The contrast images and/or non-contrast images may be 2D images, such as fluoroscopic images.

The contrast image may be automatically detected by optical based approaches, for example, a video icon indicating contrast injection appearing on the 2D images (e.g., fluoroscopic images) may be detected.

The computed annulus line may be presented on the contrast image. Optionally, an operator (e.g., the physician) may confirm the location of the annulus line, and/or correct the location. Optionally, the method may include requesting the operator (e.g., the physician) to confirm the location of the annulus line, e.g., by a pop-up on the screen.

At 904, a spatial relationship between the annulus line and a detected fiducial object (detected in the same image used to detect the annulus line) may be computed. The fiducial object may be, for example, an accessory catheter located in proximity to the native valve, such as a pigtail catheter that may be used for the injection of contrast. Alternatively or additionally, the fiducial object comprises one or more anatomical features of the subject, for example, a feature of the native aortic valve, a feature of the left ventricular outflow tract (LVOT), a feature of a coronary artery. The anatomical feature(s) may be detected, for example, by a trained ML model.

The spatial relationship may be computed between the annulus and a center of mass of the fiducial object, e.g., the accessory catheter.

The spatial relationship may be, for example, one or more distances (e.g., in pixels and/or millimeters), and/or one or more vectors that indicate relative direction, and/or a geometric shape of a defined size (e.g., triangle having a vertex at the center of mass and a base of the annulus line), and/or a mapping (such as a mapping function).

Alternatively or additionally, the spatial relationship may be computed by projecting the fiducial object to the structure, optionally the annulus line.

At 906, the anatomical structure, optionally the annulus line, is automatically detected by analyzing a target image that excludes injected contrast (also referred to herein as a non-contrast image). The target image that excludes injected contrast may be captured after the contrast image that depicts contrast was captured, i.e., after the contrast has dispersed.

The anatomical structure, optionally the annulus line, may be automatically detected by detecting the fiducial object within the target image that excludes contrast, and applying the spatial relationship. For example, the center of mass of the fiducial object is computed from the target image, and a vector implementation of the spatial relationship is used to map the location of the center of mass to the annulus line.

At 908, the detected anatomical structure is presented. Optionally, the detected annulus is presented on a display. The detected annulus may be presented as a visual overlay over the target image on a display.

At 910, one or more features described with reference to 906 and 908 may be iterated, for dynamically tracking the location of the anatomical structure, for example the annulus line, on subsequent non-contrast images. The tracked annulus line may be used, for example, for guiding location of deployment of a prosthesis heart valve.

Optionally, the annulus line, detected as described with reference to 902, represents the annulus line prior to an event that disrupts the annulus, for example, inflation of a balloon within the native valve (e.g., valvuloplasty) such as to crack calcifications and/or adhesions to increase the area of the native valve to enable deployment of the prosthetic heart valve. The annulus line may be detected after the event, using the spatial relationship computed prior to the event, as described with reference to 906. The annulus line may be detected after the event using the same spatial relationship computed prior to the event, since the event disrupts the leaflets and/or calcifications adhering to the leaflets and surrounding tissues, but the annulus line remains substantially unaffected.

Referring now back to FIG. 8, image 1002 (e.g., fluoroscopic images) depicts annulus line 1004, fiducial object 1006, and spatial relationship 1008. Annulus line 1004 is computed as described herein, for example, a line drawn between a first nadir of a first cusp 1010A and a second nadir of a second cusp 1010B of the aortic valve. Fiducial object 1006 may be for example, a pig tail catheter. The center of mass of the catheter may be computed and used. Spatial relationship 1008 is computed, for example, as a triangle between the center of mass of fiducial object 1006 and annulus line 1004.

Image 1002 is shown with contrast 1012 within the aortic root. Once spatial relationship 1008 is computed, location of annulus line 1004 may be computed using fiducial object 1006 for images that exclude contrast 1012, where annulus line 1004 cannot be directly visualized.

Referring now back to FIG. 9, features described with reference to FIG. 9 may be implemented with, replaced by, combined with, and/or used in addition to, other features of other methods described herein.

At 1104, 2D image(s) (e.g., fluoroscopic image(s)) of the subject (also referred to herein as target images) may be taken when the subject is injected with contrast during the intervention. The contrast may injected into the aortic root, such as during a TAVI procedure.

The target image depicting contrast may be referred to herein as a contrast enhanced image.

The target image may depict a valve prosthesis device in the subject. The valve prosthesis device may be in the compressed state (i.e., prior to deployment), in the partially expanded state (i.e., during deployment), and/or in the expanded state (i.e., fully deployed).

At 1106, a first anatomical structure in the target image may be automatically detected. The first anatomical structure may be visible in the contrast enhanced image, and not visible (or difficult to visualize) without contrast.

Examples of first anatomical structures include: the annulus line, a lower annulus of a subject's native aortic valve, middle of a subject's native aortic valve, top of a subject's native aortic valve, one or more commissures of a subject's native aortic valve, the leaflets of a subject's native aortic valve, the sinuses next to a subject's native aortic valve.

The first anatomical structure may be automatically detected by feeding the target image into a machine leaning model trained on a training dataset of sample images labeled with a ground truth indicating the first anatomical structure.

Alternatively, or additionally, the first anatomical structure may be automatically detected by processing the target image by cross correlation the target image with the 3D image, and mapping the first anatomical structure found in the 3D image to the target image. A projection of the 3D image to a 2D plane is computed, were the 2D projection of the 3D image is registered with the 2D target image. The projection may be computed, for example, using the pose of the image sensor that captured the 2D image. The pose of the image sensor may be found, for example, using a pose sensor, and/or by optical character recognition to extract text of the target image indicating the pose of the image sensor. The first anatomical structure may be found on the 2D projection, and mapped to the target image using the registration.

At 1108, optionally, another image (e.g., 2D fluoroscopic image) is obtained when no contrast was injected (also referred to herein as a non-contrast image), such as after the injected contrast has dissipated.

At 1110, a second anatomical structure associated with the native valve may be automatically detected in the non-contrast image. The second anatomical structure, which cannot be visualized and/or is difficult to visualize in the image with contrast. The second anatomical structure may be visible and/or better visualized in images without contrast.

The second anatomical structure may be located within the native valve of the subject.

Examples of second anatomical structures include: calcification signature (i.e., pattern of calcification deposits), tissue thickening, and blood flow derived dynamic changes.

At 1112, a fiducial object, located in both the non-contrast image(s) and the contrast image(s), may be automatically detected.

The fiducial object may be visible in the non-contrast image(s) and in the contrast image(s).

Optionally, the fiducial object is a catheter located in the aorta of the subject, for example, the catheter used to administer the contrast (e.g., pigtail).

Referring now back to FIGS. 10, 2D non-contrast fluoroscopic image 1202 depict calcification deposit (i.e., calcification signature) 1204 and pigtail catheter 1206 (i.e., fiducial object).

Referring now back to FIGS. 11, 2D contrast enhanced fluoroscopic image 1302 depicts annulus contour 1304, and pigtail catheter 1306 (i.e., fiducial object). Calcification deposit (i.e., calcification signature) 1308 is difficult to visualize, and is usually not visible.

Referring now back to FIG. 9, at 1114, the first anatomical structure may be automatically co-located to the second anatomical structure. The automatic co-locating of the first anatomical structure to the second anatomical structure may be based on an identified spatial relationship between the first and second anatomical structures.

The first anatomical structure may be detected without requiring presence of the fiducial object in the image. The first anatomical structure may be detected using the combined mapping, which was computed using the presence of the fiducial object. The fiducial object may be moved and/or removed after the combined mapping has been computed.

Optionally, the co-locating is done by computing a first sub-mapping between the first anatomical structure and the fiducial object. The sub-mapping may be, for example, a spatial relationship, one or more distances (e.g., in pixels and/or millimeters), one or more vectors that indicate relative direction, a geometric shape of a defined size (e.g., triangle having a vertex at the center of mass and a base of the first anatomical structure), and/or a mapping function. A second sub-mapping between the second anatomical structure and the fiducial object depicted may be computed. A combined mapping between the first structure and the second structure may be computed according to a combination of the first sub-mapping and the second sub-mapping.

Optionally, the first mapping and the second mapping are computed for a pair of the non-contrast image and the contrast enhanced image captured at a same pose of an image sensor, same subject orientation, and same zoom.

Alternatively or additionally, the spatial relationship between the first and second anatomical structures may be calculated from the 3D image (e.g., CT or MRI) of the subject taken, optionally take prior to the intervention, for example, as described herein.

At 1116, subsequent non-contrast images may be obtained.

At 1118, the first anatomical structure, which is difficult to visualize and/or cannot be visualized on non-contrast images, may be detected on the non-contrast image(s).

The first anatomical structure may be detected by applying the co-location to the second anatomical structure, which may be automatically detected on the non-contrast images. For example, the calcification signature is automatically detected on the non-contrast image, while the annulus line cannot be visualized on the non-contrast image.

The combined mapping may be applied to the detected second anatomical structure to detect the location of the first anatomical structure on the non-contrast image.

In an exemplary implementation, the first structure is a contour of an annulus of the native valve, the second structure is calcium deposits (i.e., calcification signature) on the valve, and the fiducial object is a catheter (e.g., pigtail) located in the aorta.

Alternatively or additionally, the location of the first anatomical structure on the non-contrast image is obtained as an outcome of feeding a combination of the non-contrast image(s) and the contrast image(s) into the machine learning model. The machine learning model may be trained on a training dataset of multiple records. Each record may include a combination of sample non-contrast image(s) that depict the structure associated with the valve (e.g., second structure) and optionally the fiducial object, a corresponding sample contrast image(s) that depicts the first anatomical structure (e.g., annulus line) and optionally the fiducial object, and a ground truth indicating of the first anatomical structure on the non-contrast image(s).

The ML model may be dynamically trained using a dynamically created training dataset for the specific patient, where the ground truth is computed using the co-location, such as the combined mapping. Alternatively or additionally, the ML model is previously trained using images from different subjects.

At 1120, the location of the annulus line (or other first anatomical structure) may be dynamically tracked on subsequent images without contrast. The tracking may be done, by mapping the location of the annulus line detected on a baseline image without contrast (e.g., computed as described with reference to 1112) to a subsequent image without contrast, based on a registration between the baseline image and subsequent image. For example, computing optical flow between the baseline image and the subsequent image, and applying the optical flow to map the annulus line from the baseline image to the subsequent image. The mapping from the baseline image to the subsequent image may be computationally efficient in comparison to re-computing the combined mapping for each new image, which may enable real-time tracking.

Alternatively or additionally, pairs of sequentially obtained non-contrast images are fed to a machine learning model for tracking a location of the annulus line. The pairs of images may include the initial image with identified annulus line, and subsequent image without identified annulus line, both of which may be non-contrast images. The ML model may be the dynamically trained ML model, and/or another ML model trained on a training dataset of records, where a record includes a pair of a sample initial image with detected annulus and a subsequent image without detected annulus, and the ground truth is the subsequent image with marked detected annulus.

At 1122, the co-located first anatomical structure (e.g., annulus line) may be presented on a display, for example, as a visual overlay over the target image without contrast.

At 1124, a change in settings may be detected, for example, a change in at least one of the zoom, subject orientation, and the pose of the image sensor, may be detected.

The detected change may trigger a re-computation of the combined mapping (e.g., as in 1114). The new combined mapping may be used for subsequent images at the changed settings.

Alternatively or additionally, the combined mappings may be adapted according to the detected change, for example, by computing a transformation mapping to apply to the combined mappings where the transformation mapping is from the previous settings to the new settings.

Alternatively or additionally, detected change is fed to the machine learning model in combination with the non-contrast image.

At 1126, one or more features described with reference to 1104-1124 are iterated while the valve prosthesis device is transported on its way to be implanted in a native valve and/or while the valve prosthesis device is being positioned and/or deployed.

Referring now back to FIG. 12, features described with reference to FIG. 12 may be implemented with, replaced by, combined with, and/or used in addition to, other features of other methods described herein.

The approach described with reference to FIG. 12 tracks the annulus line on non-contrast 2D image(s) where the annulus line cannot be seen and/or is difficult to detect. The method is described in reference to annulus line tracking but may be used to track other anatomical structures which may be required as part of medical procedure. The method may be used for other procedures which require tracking of other anatomical structures which are not visible or not sufficiently visible on non-contrast images.

Referring now back to FIG. 12, at 1402, a 3D image of the subject may be accessed. The 3D image may be captured pre-procedure, for example, a CT and/or MRI scan. The 3D image may depict a native valve of the subject for transcatheter replacement in the subject, and/or other anatomical structures which require tracking in other procedures, optionally where the anatomical structures are not visible or not sufficiently visible in non-contrast images.

The native valve of the subject may be, for example, a native aortic valve, a tricuspid valve, and a pulmonary valve.

The subject may be prepared for performance of the medical intervention, for example, a transcatheter aortic valve replacement procedure.

The subject may be undergoing a medical intervention, such as a transcatheter valve replacement procedure for replacement of the native valve.

At 1404, anatomical features are identified on the 3D image. Exemplary anatomical features include calcification signature (e.g., calcification deposits), and/or an annulus plane. The annulus plane may be computed by detecting three nadirs of the three cusps of the aortic valve. The annulus plane intersects the three nadirs.

The pattern of calcification deposits may be referred to herein as a calcium signature. The calcium may be deposited in multiple clumps, which may be “randomly” distributed, in the sense that each patient is expected to have their own unique pattern of distribution of calcium deposits.

The calcification signature in the 3D image may be projected to a 2D plane which represents a template. The template may be matched to 2D images captured during the procedure, for example, in real time or near real time. The template may be of a pattern of calcification signature that may include multiple calcium deposits, within the leaflets, annulus, native valve, heart muscle, aortic wall, and/or other nearby tissues. The template may include tissues that are not impacted by an event, such as inflation of a balloon within the native valve (e.g., valvuloplasty), to enable performing the registration after the event.

The calcification signature may be detected, for example, according to a voxels having Hounsfield values within a range that defines calcium.

The pre-procedure image may be used for generating a template which may be registered (e.g., substantially matched and/or correlated) with a signature of a 2D image (e.g., fluoroscopic image) obtained during the procedure. The registration between the 2D image obtained during the procedure (e.g., in real time or near real time) and the 3D image (e.g., obtained pre-procedure) enables mapping anatomical features found in the 3D image to the 2D image, even when the anatomical features are not visible or not sufficiently visible on the 2D image.

At 1406, a 2D target image is obtained during the procedure, optionally in real time or near real time. For example, a 2D image depicting a native valve of a subject. The 2D target image may be non-contrast. The annulus line (or other structure) being detected and/or tracked is not visible and/or not visible well on the non-contrast image.

At 1408, the calcification signature in the 2D target image is detected, for example, by thresholding pixel intensity values to values indicating calcium, machine learning approaches, edge detection, and the like. In other embodiments, other anatomical features are identified on the 2D image.

Optionally, the calcification signature is identified on an anatomical structure(s) that shares similar displacement during cardiac and/or breathing cycles with the annulus line.

Optionally, the calcification signature is identified in tissues that are not significantly impacted by a balloon inflated within the native valve. For example, inflation of the balloon displaces the calcification of the leaflets. This allows using the same calcification signature before and after inflation of the balloon.

At 1410, a calcification template is selected from multiple calcification templates of a 3D image of the subject according to a pose of the image sensor that captured the 2D target image.

Optionally, the calcification template is computed by projecting the 3D image with the annulus plane to a 2D plane having the pose of the image sensor.

Alternatively or additionally, the calcification template is computed by searching the calcification signature for a 2D calcification template of the 3D image according to a correlation requirement. Optionally, multiple 2D projections of the 3D image are pre-computed according to predicted poses of the image sensor. The search may be done using the pre-computed 2D projections, to find the closest matching projection. For example, the 2D projection with highest correlation to the calcification signature of the 2D target image.

The annulus plane (or other structure in 3D) is mapped to an annulus line (or 2D projection of the 3D structure) on the calcification template. For example, the annulus plane in the 3D image is projected to a 2D annulus line during the projection of the 3D image to the 2D plane of the calcification template.

At 1412, the 2D target image may be registered to the calcification template. Registration may be performed, for example, by matching features, and/or other approaches for image registration.

At 1414, the annulus line (or other structure) depicted in the calcification template may be mapped to the registered 2D target image.

At 1416, the annulus line is provided, optionally presented on the 2D target image such as an overlay.

At 1418, one or more features described with reference to 1404-1416 may be iterated. For example, for iteratively tracking the annulus line on subsequent images. Alternatively or additionally, the annulus line may be tracked on subsequent non-contrast 2D image by dynamically correcting for the pose of the image sensor. For example, computing a transformation between the previous pose of the image sensor and the new pose of the image sensor, and applying the transformation to the annulus line of the previous image to obtain the new location of the annulus line on the new image.

Referring now back to FIG. 13, the flowchart of the process for computing the distance between the annulus line and the distal end of the THV, is described.

At 1502, a target image, optionally non-contrast, optionally 2D, is accessed. The image may be a fluoroscopic image obtained during a TAVI procedure.

The target image may depict a partial expansion of a transcatheter heart valve (THV) positioned within the native aortic valve of the subject.

At 1504, an annulus line (also referred to herein as a lower annulus) of the subject's native aortic valve may be automatically detected by analyzing the target image.

Various approaches may be used for detecting the annulus line, for example, as described herein, such as with reference to 208 of FIG. 2, FIG. 7, FIG. 9, and FIG. 12. Other exemplary approaches are now described.

Optionally, a candidate location of the lower annulus of the native aortic valve is detected by analyzing sample image(s) depicting the lower annulus of a native aortic valve that excludes presence of the THV within the native aortic valve. The sample image(s) may include contrast. The candidate location may be detected and/or learned from the sample image(s) of the subject obtained prior to placing the THV in the native aortic valve. Alternatively or additionally, the candidate location is learned from sample image(s) of sample individual(s) and/or of a generic model. The sample image may be registered with the target image (which depicts the partially expanded THV within the native aortic valve). The candidate location of the sample image(s) may be mapped to the target image to obtain the lower annulus of the target image. The mapping may be performed by computing a transformation from the sample image to the target image, and applying the transformation to the candidate location for obtaining the lower annulus. The sample image(s) may include injected contrast located within the cusps of the aortic valve and the ascending aorta. The target image may exclude injected contrast within the cusps of the aortic valve and the ascending aorta. The lower annulus detected in the sample image(s) may be mapped to the target image for identifying the lower annulus in the target image.

At 1506, a distal end of the partially expanded THV may be automatically detected by analyzing the target image. The analysis may be performed by feeding the target image into a machine learning model trained on a training dataset of records, where each record includes a sample image of the partially expanded THV, and a ground truth indicating the distal end thereof. In another example, the analysis may be performed by image processing and/or computer vision approaches, for example, comparing the target image to a template depicting the distal end of the partially expanded THV. A correlation may be computed between the target image and the template. The distal end of the partially expanded THV may be detected when the correlation is above a threshold.

Exemplary details of ML models for detecting the distal end of the partially expanded THV and/or of the lower annulus are now described.

Optionally, the ML model is trained on a training dataset of records, where each record includes a sample image of a sample individual or of a generic model labelled with a ground truth label indicating a lower annulus of a native aortic valve and/or a distal end of a partial expanded THV for positioning within the native aortic valve. The ML model may be dynamically further trained on images of a subject depicting the lower annulus of the native aortic valve, to obtain a personalized ML model. The target image of the subject may be dynamically fed into the personalized ML model to obtain a detected lower annulus and a detected distal end of the partially expanded THV.

Optionally, the annulus line may be detected independently of the THV (e.g., as described herein). The THV may be detected using other approaches, for example, using an ML model, image processing, and/or computer vision based approaches. For example, the ML model for detecting the THV may be training on a training dataset of records, where each record includes a sample image of a sample THV, optionally in a compressed state and/or in various states of deployment (e.g., partial deployment, full deployment). In another example, image processing and/or computer vision based approaches used to detect the THV include for example, detecting an edge of the THV, detecting a pattern of pixel intensity values unique to the THV, and/or correlating with a template depicting the THV.

Optionally, a record(s) used to train the ML model include multiple images captured over multiple phases of a cardiac cycle and/or a breathing cycle of the subject.

Optionally, a personalized mask excluding a region around the lower annulus and/or the distal end of the partially expanded THV is computed. The mask may be applied to the sample image of the record used to train the ML model and/or to the target image(s) fed into the ML model. The mask may remove irrelevant data from the image(s) which may help the ML model to avoid focusing on irrelevant features.

Optionally, contrast enhanced image(s) of the subject depicting injected contrast located within the cusps of the aortic valve and the ascending aorta, are automatically analyzed to identify the lower annulus, for example, as a contour of the contrast and/or using ML model approaches, and/or other approaches described herein. The identified lower annulus may be automatically labelled in the contrast enhanced image(s). A mask over the contrast enhanced region of the contrast enhanced image that includes the cusps and/or the ascending aorta may be applied to define a masked region. A synthesized image may be created by replacing the masked region of the contrast enhanced region with values of pixels corresponding to the masked region in a non-contrast enhanced image. The record including the synthesized image labeled with the identified lower annulus may be used for training the ML model for detecting the lower annulus in response to an input of a non-contrast enhanced image.

At 1508, the device depth of the THV is computed. The device depth is a distance between the lower annulus and the distal end of the THV.

The term device depth and distance are used interchangeably.

Optionally, the detected lower annulus is represented as a first line representing a plane of the lower annulus. The detected distal end of the THV is represented as a second line representing a plane of the distal end of partially expanded THV. The distance is computed between the first line and the second line.

Alternatively or additionally, a fiducial marker of the THV having a known dimension(s) is automatically detected. A calibrated distance is computed by calibrating the distance according to the known dimension. The fiducial marker may include, for example, a strut or portion thereof of a scaffold of the THV that remains the known fixed dimension throughout expansion of the THV. In another example, the fiducial marker may include a catheter (pigtail used to inject the contrast) located in the aorta have a fixed dimension such as of a marker.

At 1510, an automatic determination may be made whether the distance is within a target range indicating correct positioning of the THV within the native aortic annulus, optionally at the annulus line. The target range may be defined according to an identifier of the THV. For example, different THV may have different identifiers and different target ranges.

At 1512, one or more data items may be provided, optionally presented on a display, optionally within a graphical user interface (GUI). For example, the distance and/or the determination may be presented.

Optionally, an overlay over the target image that visually marks the lower annulus and/or the distal end may be provided.

Optionally, a visual and/or an audio indication of when the distance is within the target range may be presented. The visual indication may be presented, for example, in the form of a binary or tertiary indication, such as three colors. A first color indicates that the distance is within the target range. A second color indicates that the distance is external to the target range, optionally in a certain direction. For example, that the distance is lower than the target range. A third color may indicate that the distance is external to the target range in the other direction, for example, that the distance is greater than the target range. In another example, a coded bar is presented, representing the possible values of the distance, from low to higher. An indication on the coded bar represents the current distance. A region of the coded bar may indicate the target range.

Optionally, when no distal end of the partially expanded THV is detected, a visual and/or an audio indication of where to position a distal end of the THV in the compressed state for expansion of the THV such that the distal end of the THV when expanded is predicted to be located within the target range, may be presented.

At 1514, when the distance is external to the target range, the operator may be instructed to move the THV to another location.

At 1516, one or more features described with reference to 1502 to 1514 are iterated for tracking the device depth.

Optionally, when the THV is a self-expanding THV, and the distance is monitored over sequential target images during the self-expansion of the THV.

Optionally, the candidate location and the lower annulus are detected and tracked over sequential sample images and target images by matching phases of breathing cycle(s) and/or at least one cardiac cycle(s) depicted in the sample images to corresponding phases of the target images.

At 1518, when the distance is within the target range, the THV may be deployed, for example, for treating subject for a dysfunctional aortic valve by implanting the THV within the native aortic valve.

Additional features described below may be implemented with embodiments described above, for example, as described with reference to FIGS. 1-19.

An aspect of the present invention includes a method of guiding a trans-catheter aortic valve replacement intervention in a patient.

Trans-catheter aortic valve replacement (TAVR) is a minimally invasive heart procedure to replace a thickened aortic valve that can't fully open, a condition known as aortic valve stenosis. The aortic valve is located between the left ventricle and the aorta. If the valve doesn't open correctly, blood flow from the heart to the body is reduced.

TAVR can help restore blood flow and reduce the signs and symptoms of aortic valve stenosis—such as chest pain, shortness of breath, fainting and fatigue.

Trans-catheter aortic valve replacement may also be called trans-catheter aortic valve implantation (TAVI).

As used herein, guiding an intervention may include providing information about the manner at which the intervention proceeds. For example, the information may include images of the interior of the patient's body, where intervention takes place or where a device used by the operating surgeon is navigating. The information may be provided to the operating surgeon or to any other member of the operating stuff.

The disclosed method can be implemented only on a computer. The computer typically includes a processor and a digital memory that stores instructions, that when executed by the processor cause the processor to carry out a method as described herein. In some embodiments, the computer may also include a display for displaying information for guiding the intervention.

The method includes obtaining fluoroscopic 2D images capturing an aortic valve prosthetic device in the aorta of the patient, e.g., in the descending aorta of the patient. The prosthetic device may be, for example, Evolute™ by Medtronic, Porticco™ by Abbott, or LOTUS Edge™ or NEO™ by Boston Scientific.

These and other aortic valve prosthetic devices have to be oriented in alignment with the native commissure of the native heart valve, and include markers that should help the operating surgeon implanting the prosthetic device at the correct orientation. In some of the devices the marker includes a plurality of marking units, e.g., radiopaque dots or short lines, that are oriented one in respect to the other in some predetermined manner when, and only when, the device is properly oriented.

However, the markers are frequently hard to find in the image, and the operating surgeon is required to invest considerable cognitive resources for finding it in the image, and determining the state of the marker, which is indicative to the orientation being proper or not.

It is noted that the appearance of the marker in the 2D image depends not only on the way the marker is aligned in respect to the patient, but also on the positioning of the imager. Therefore, it is not necessarily sufficient to identify how the marker appears, but also the viewing angle at the image was taken. Usually, the implantation of the prosthetic valve device is carried out in a certain, preferable, fluoroscopic view (i.e., “cusp overlap view”).

In some embodiments, if the view is identified not to be the preferable (e.g., cusp overlap) view, the methods or apparatuses alert that the view is not the preferable one. In some embodiments, an indication of the of the annulus line of the heart of the subject also be provided even if the image is obtained with an imager at non-preferable position.

The fluoroscopic images may be obtained from an imager that is integral to the guiding device, or from an independent imager. In some embodiments, the guiding computer is directly connected to the image sensor, and receives as input data from the image sensor. Optionally, these data are the same used by the imager for producing and displaying the fluoroscopic image on the imager display. In some embodiments, the computer may include (or may receive input from) a camera that photographs the image sensor display, and the 2D images are obtained from this camera for purpose of the guiding.

The fluoroscopic images are preferably obtained and processed online, so that the operating surgeon may receive the guidance in real time, although in some embodiments, the guiding may be provided after the fact, for post hock analysis and staff education.

At least one of the obtained fluoroscopic images is fed to a machine-learning model trained to identify the annulus line of the heart of the subject and output an indication to of the annulus line of the heart of the subject.

In some embodiments, the training is with images labeled by human experts. The labeling includes a label of the position of the annulus line of the heart of the subject. In some embodiments, the machine-learning includes two modules, one trained to identify the template of calcification signature of the subject in the image, and another trained to identify the of the annulus line of the heart of the subject. Calcium signature may also refer to herein as calcification signature.

The images used for training may include images received from the imager, and in addition, the same images transformed randomly for rotation, zoom, and/or shift. In some embodiments, each image is transformed for up to 100 additional times, so with 300 images the training is actually happening on 30 000 slightly different images which may decrease the risk of overfitting. This way the amount of the images used in the training may be increased without having to collect and label addition images. Optionally, the training is done using stochastic optimization, and each epoch a different set of the images is used in the training.

The output from the machine-learning model may include indication to the position of the annulus line of the heart of the subject in the image The output may be received by a computer module that controls a display to indicate the position of the annulus line of the heart of the subject, for example: the annulus line may be highlighted on the image; or otherwise marked as an overlay over the image. In some embodiments, the display may be visual. e.g., a textual message may appear on the display, or an indicator may be lighted with lights of different colors, etc.

In some embodiments, the method is repeated with new images obtained from the imager as the operation proceeds. In such embodiments, from time to time (e.g., every 0.2, 0.5, 1, 2 or 5 seconds), a new image is fed into the machine-learning model to detect the annulus line or another structure of a native valve. The annulus line may be displayed to an operator continuously during the procedure. This may enable to physician to implant the valve prosthesis device at the correct location.

Referring now back to FIG. 14, it is noted that some features are optional. Some features described herein may be implemented in combination with, and/or in replacement of, and/or in additional to, features described with reference to FIGS. 2, 7, 9, and 12. For example, features 204, 205, 208, and 212 of FIG. 2 may be implemented based on features described with reference to FIG. 14, or vice versa. In another example, the spatial relationship of 904 of FIG. 9 may be implemented based on 1616 of FIG. 14. In yet other example, features 1104-1126 of FIG. 9 may be implemented based on features described with reference to FIG. 14, or vice versa. In yet another example, features 1402-1418 of FIG. 12 may be implemented based on features described with reference to FIG. 14, or vice versa

At 1602, a fluoroscopic image is accessed. The fluoroscopic image depicts at least an aortic root of the subject, which includes at least the native aortic valve. A guidewire and/or aortic valve prosthesis device may or may not be depicted in the fluoroscopic image. The fluoroscopic image may depict injected contrast or may exclude injected contrast.

Optionally, each frame of a video stream generated by an image sensor (e.g., fluoroscopy machine) is grabbed. The frame represents the fluoroscopic image described herein. Alternatively, every few frames may be grabbed.

A frame rate may be defined to be higher than a fluoroscopy frame rate. A resolution may be defined higher than a fluoroscopy native resolution.

Additional details and/or examples of accessing fluoroscopic images are provided, for example, as described herein, such as with reference to 204 of FIG. 2.

Optionally, an accessory catheter is introduced into the patient, in particular to the aorta and/or native aortic valve and/or left ventricle during the TAVI procedure. The accessory catheter that may be introduced into the patient may be, for example, a catheter for injecting contrast and/or for being used as a marker, such as a pigtail catheter. In standard TAVI procedures, the pigtail catheter is introduced into the left ventricle and/or aorta (e.g., within the native aortic valve, such as within a cusp) for injection of contrast and/or to help locate the annulus. Alternatively, the accessory catheter is not introduced at all, or is used for shorter intervals and/or for fewer steps of the TAVI procedure in comparison to standard TAVI procedures.

Optionally, in at least some embodiments described herein, the accessory catheter (e.g., pigtail) is not needed to identify and/or track anatomical structure(s) of the native aortic valve, for example, the annulus line. The tracking of the annulus line may be done, for example, by identifying a 2D projection of a 3D image of the native aortic valve and surrounding tissues (e.g., obtained by a pre-procedure CT scan) where the anatomical structure(s) are indicated, registering the 2D fluoroscopic image(s) (obtained during the procedure) with the 2D projection, and mapping the location of the annulus line(s) on the 3D image, which are projected to the 2D projection, to the 2D fluoroscopic images(s).

At 1604, the fluoroscopic image is analyzed to detect whether or not contrast is depicted therein.

A label may be generated indicating the result of the analysis, for example, indicating contrast (or injection) or no label or indicating no contrast (or no injection).

Detection of contrast (e.g., injection of contrast) may be performed, for example, based on an image processing approach. Background and/or foreground analysis (e.g., learning) of sequentially obtained fluoroscopy images may be performed. When the foreground includes high intensity regions (i.e., due to the contrast material injection) in the relevant location (e.g., within the aortic root and/or aortic valve and/or ascending aorta), contrast and/or injection thereof is detected.

In response to the analysis of the fluoroscopic image indicating presence of contrast (referred to herein as a contrast fluoroscopic image), at 1606, one or more fiducial objects depicted in the at least one contrast fluoroscopic image are automatically detected.

The location of the fiducial object(s) within the contrast fluoroscopic image may be computed, for example, relative to an external coordinate system, and/or location of pixels indicating the fiducial object(s) within the image.

The fiducial object(s) may include a catheter, optionally an accessory catheter which may be designed for injection of contrast (e.g., pigtail catheter).

The accessory catheter may be detected, for example, by feeding the contrast fluoroscopic image into a catheter detection machine learning model. The catheter detection machine learning model may be trained on a training dataset of contrast fluoroscopic images depicting catheters at different locations, labelled with a ground truth indicating location of the catheter within the contrast fluoroscopic image, a bounding box encompassing the catheter, and the like. The training dataset may include images without catheters. In another example, the catheter may be detected using other image processing approaches, for example, looking for edges and/or features indicating an elongated straight object and/or a pigtail like shape, and the like.

Multiple fluoroscopic images (e.g., about 25, or 50, or 75, or other number) may be analyzed for identifying that the accessory catheter is located within a cusp of the native aortic valve. The initial frame may include a contrast fluoroscopic image, sequentially followed by non-contrast fluoroscopic images as the contrast dissipates. The location of the detected accessory catheter within the cusp may be verified by tracking motion of the location of the detected catheter between the multiple contrast fluoroscopic images, and checking that the motion corresponds to expected movement of leaflet(s) of the native aortic valve in response to heartbeats. When motion of the accessory catheter does not correlate to expected motion corresponding to motion of the beating heart, and/or when no motion of the accessory catheter is detected, the accessory catheter is determined to be located externally to the cusp of the aortic valve. The motion of the accessory catheter over multiple frames may be computed using the location of the accessory catheter in the initial contrast fluoroscopic image. Optionally, a label is generated indicating whether the accessory catheter is within the cusp or not within the cup.

At 1608, the annulus line is detected in the contrast fluoroscopic image.

Optionally, a contour of an aortic annulus is detected in the contrast fluoroscopic image.

The contour may be detected by segmenting pixels with contrast in proximity to the fiducial object (e.g., catheter). The pixels with contrast in proximity to the catheter may represent the foreground of the image.

The contour of the segmented pixels may be identified, for example, as the border of the segmented pixels. The segmented pixels as a whole may represent the aortic outflow track. Optionally the distal border of the segmented pixels, which corresponds to the leaflets and/or cusps of the native aortic valve, is identified.

At 1610, the detected contour may be overlaid on the contrast fluoroscopic image. For example, marked with a color, bolded, and the like.

The user may indicate whether the detected contour is correct or incorrect, for example, via a user interface which may present the overlaid image.

In response to the user indicating that the contour is incorrect, the method may return to 1602 for another iteration.

In response to the user indicating that the contour is correct, at 1612, the contour may be analyzed for detecting at least two nadirs of at least two cusps of the native aortic valve. Each nadir may be detected, for example, as a local minimum of the contour. The annulus line may be computed as a line connecting (e.g. intersecting) the at least two nadirs. The line may be presented in the overlay, replacing the previously presented contour.

Referring now back to FIG. 15, in schematics 1700A and 1700B, fluoroscopic images 1702 depict contrast enhanced regions 1706 denoting the aortic outflow tract. In schematic 1700A, a distal end 1708 of contrast enhanced region 1706 is overlaid with a contour 1704, representing the annulus of the aortic valve. Arrow 1760 denotes a user approving the location of contour 1704, triggering computation of annulus line 1750. Schematic 1700B depicts two nadirs 1780A-B of contour 1708, for example, as local minimums. Annulus line 1750 may be computed as a line connecting (e.g. intersecting) the two nadirs 1780A-B.

Referring now back to FIG. 14, Optionally, at 1614, one or more additional fiduciary markers may be detected and/or indicated in the overlay over the image.

The additional fiduciary marker(s) may be structures that have motion that is synchronized with the beating heart (also referred to herein as synchronized structures), for example, calcification signature indicating patterns of calcification deposits (e.g., calcified cusps, calcified arteries, calcification in muscle of the heart), and/or other tools inserted into the body, for example, a pacing wire, a guidewire (e.g., stiff guidewire), and the like.

The additional fiducial marker(s) may be other anatomical structures, for example, inferior posterior and/or anterior superior regions of the membranous septum floor, and/or the inferior part of the pyramidal space.

The synchronized structures may be obtained, for example, manually labelled by a user via a GUI presenting the fluoroscopic image and/or overlay, and/or automatically by an anatomical marker machine learning model trained on a dataset of fluoroscopic images (contrast and/or non-contrast) labelled with ground truths indicating the synchronized structures(s).

At 1616, a mapping (also referred to herein as a spatial relationship) between the detected annulus line and the fiducial object(s) is computed. The mapping may be computed between the detected annulus line and the accessory catheter when located within the cusp. Alternatively or additionally, the mapping may be computed between the detected annulus line and one or more synchronized structures.

The mapping may be, for example, a vector indicating where the annulus line is to be located in a 2D image given a detected location of the fiducial object(s). The vector may include a vertical component and a horizontal component, along the x-axis and y-axis. Optionally, the length of the vector and/or components is according to a scale of the fluoroscopic image, for example, a defined distance represented by individual pixels of the fluoroscopic image.

At 1618, the fluoroscopic image may be registered with a pre-procedure 3D image, for example, a CT scan obtained prior to the session in which the fluoroscopic images are captured. The registration may be between the contour of the annulus (also referred to herein as an annulus silhouette) seen on the fluoroscopy with the annulus segmented from the pre-procedure CT.

The accuracy of the registration may be increased by further registering additional fiduciary markers that are depicted in both the fluoroscopy image and the pre-procedure 3D image, for example, calcification signature.

The registration may enable tracking the annulus line in response to projection changes and/or zoom changes of the fluoroscopy system and/or in response to changes in orientation of the subject, for example, as described with reference to 1624.

Referring now back to 1604, alternatively when no contrast is detected in the fluoroscopic image, at 1620, a verification that the annulus line was detected (e.g., as described with reference to one or more of 1606-1614) and/or that the mapping was computed (e.g., as described with reference to 1616) may be performed. In response to no annulus line having been previously detected and/or no mapping computed, the method may return to 1602 for another iteration. The method may return to 1602 for obtaining a contrast fluoroscopic image computing the annulus line and/or the mapping.

At 1622, one or more synchronized structures may be detected. The location of the synchronized structures within the image may be detected.

The synchronized structures may be detected in a sequence of multiple fluoroscopic images obtained over at least one heart beat (e.g., cardiac cycle). The synchronized structures may be detected by analyzing positions of pixels denoting the synchronized structure(s) over the multiple fluoroscopic images, to determine whether the movement of the pixels is synchronized with the moving heart while it is beating. For example, comparing a trajectory of motion of the synchronized structure(s) to a predefined trajectory representing the beating heart, where the individual synchronized structures(s) are determined by a detector machine learning model trained on images labelled with locations of the synchronized structure(s) and/or other image processing approaches such as pixels having an intensity greater than a threshold indicating calcification. The predefined trajectory may be defined, for example, based on an analysis of previously obtained images of a beating heart. In another example, synchronized structures may be detected by feeding the sequence of images into a synchronization machine learning model training on sequences of images labelled with a ground truth indicating location of the synchronized structures and/or whether the images depict synchronized structures or not.

The synchronized structures may be used, for example, in a case when the image does not depict the accessory catheter (e.g., pigtail) being not located within a cusp of the native aortic valve. For example, there is no accessory catheter, or the accessory catheter is external to the cusp.

Alternatively, when the image does depict the accessory catheter within the cusp, the synchronized structures may not be detected, or may be detected and used to further increase accuracy of the annulus line detection.

At 1624, a change in at least one of the zoom of the fluoroscopic image, orientation of the subject depicted in the fluoroscopic image, and the pose of the image sensor that captures the fluoroscopic image, may be detected.

The change may be detected, for example, by applying an optical character recognition (OCR) to the fluoroscopic image, and extracting a value of the projection of the fluoroscopy machine that is automatically added to the fluoroscopic image. The change may be detected by other approaches, for example, monitoring user interactions with the image sensor (e.g., fluoroscopic machine) and/or interface of the image sensor (e.g., fluoroscopic machine), such as by monitoring the interface, and/or by sensors attached to the image sensor (e.g., fluoroscopic machine) that sense a change in orientation, and/or by sensors attached to the subject that sense a change in orientation, and/or by analyzing the images themselves.

As described with reference to 1618, the annulus line detected on the fluoroscopy image may be registered to the 3D annulus segmented from the pre-procedure 3D image. In response to the detected change, a transformation of the 3D annulus segmented from the pre-procedure 3D image is computed according to the detected change. Optionally, a first projection of a 3D annulus in the 3D image to a first 2D plane corresponding to an orientation of the image sensor that captures fluoroscopy images is computed. In response to the change, a second projection of the 3D annulus to an annulus line on a second 2D plane corresponding to the changed orientation of the image sensor is computed. The projected annulus line is presented on the overlay.

Alternatively or additionally, in response to the change, the current fluoroscopic image captured after the change is aligned to a preceding fluoroscopy image obtained prior to the change, for example, according to optical flow analysis. The alignment may compensate, for example, for movement of the table on which the subject is located, and/or zoom of the image sensor. The alignment may maintain the registration between the 3D image and the annulus of the fluoroscopic image.

Referring now back to FIG. 19, annulus plane 2104 of left ventricle 2102 is computed and/or segmented from 3D image 2100. 3D image 2100 may be obtained as a pre-procedure image, obtained prior to the current procedure where fluoroscopic images are being captured. 3D image 2100 may be extracted, for example, from a CT scan, MRI scan, and the like. Annulus plane 2104 may be computed by identifying the three nadirs of the three cusps of the aortic valve. One nadir 2106 is marked for clarity (the other two nadirs are present but not marked). Annulus plane 2104 may be a two dimensional data structure, represented in a 3D space, that fits to the regions (e.g., voxels, points) corresponding to the three nadirs, such as intersecting the three nadirs. It is noted that 3D image includes at least the aortic valve and/or aortic root, to enable identifying the three nadirs. As shown, annulus plane 2104 is drawn as a 2D circle in the 3D space having a circumference that is best fitted to the regions corresponding to the three nadirs. Annulus plane 2104, which is represented in the 3D space, may be projected to a 2D plane corresponding to a current orientation of the C-arm of the fluoroscopic machine that captured the current fluoroscopic image, for generating a 2D representation of the annulus line. The projected 2D representation of the annulus line is associated with a location within the 2D fluoroscopic image. The generated overlay, which is overlaid over the fluoroscopic image captured after the change, may include the 2D annulus line computed by projecting the 3D annulus plane to the 2D plane.

At 1626, the non-contrast fluoroscopic image(s) may be analyzed for detecting the location of one or more fiducial objects, including the accessory catheter (e.g., pigtail) and/or synchronized structures, for example, as described with reference 1606.

In response to detecting the accessory catheter, at 1628, the non-contrast fluoroscopic image may be analyzed to detect whether the accessory catheter is located in a cusp of the aortic valve, for example, as described with reference 1606.

The detection of the accessory catheter within the cusp and/or or synchronized structures that vary position according to the beating heart may be used for mapping to the annulus line, based on the computation of the mapping line which was made using the same accessory catheter and/or synchronized structures.

In response to determining that the accessory catheter is located within the cusp, at 1630, a location of the annulus line is computed relative to the accessory catheter within the cusp by mapping the location of the accessory catheter to the location of the annulus line. The mapping was previously computed using the location of the accessory within the cusp, as described with reference to 1616 may be used.

At 1632, an overlay over the non-contrast fluoroscopic image is computed. The overlay includes the annulus line overlaid over the location computed using the mapping, with reference to the detected location of the accessory catheter.

In the case of multiple sequential fluoroscopic images used to detect the location of the accessory catheter (e.g., for detecting whether the accessory catheter is located within the cusp), the location of the annulus line may be computed relative to the location of the accessory catheter as shown for example in an initial contrast fluoroscopic image of the sequence of fluoroscopic images which may include non-contrast fluoroscopic images.

Referring now back to FIG. 16, exemplary non-contrast fluoroscopic image 1802 is shown, depicting detected accessory catheter 1804 and the overlay of annulus line 1806. The location of annulus line 1806 is computed by applying the previously computed mapping to the detected location of accessory catheter 1804.

Referring now back to FIG. 14, the method may iterate back from 1602, for dynamically updating the overlay for tracking the location of the annulus line.

Alternatively to the accessory catheter not being detected (e.g., as described with reference to 1626) and/or to the accessory pigtail determined to be located externally to the cusp (e.g., as described with reference to 1628), at 1634, a location of the annulus line is computed relative to the synchronized structure(s) by mapping the location of the synchronized structure(s) to the location of the annulus line. The mapping was previously computed using the location of the synchronized structure(s), as described with reference to 1616 may be used.

In the case of multiple sequential fluoroscopic images used to detect the location of the synchronized structure(s) (e.g., for tracking movement of the synchronized structures), the location of the annulus line may be computed relative to the location of the synchronized structure(s) as shown for example in an initial contrast fluoroscopic image of the sequence of fluoroscopic images which may include non-contrast fluoroscopic images.

At 1636, an overlay over the non-contrast fluoroscopic image is computed and optionally presented on a display to the user. The overlay includes the annulus line overlaid over the location computed using the mapping, with reference to the detected location of the synchronized structure(s).

Referring now back to FIG. 17, exemplary non-contrast fluoroscopic image 1902 is shown, depicting detected synchronized structure, in particular calcification signature 1904 and the overlay of annulus line 1906. The location of annulus line 1906 is computed by applying the previously computed mapping to the detected location of calcification signature 1904.

Referring now back to FIG. 14, at 1650, optionally, the user may validate the location of the annulus line. The approval of annulus line may be used for validation of the automatically detected annulus line.

The annotation of annulus line (i.e., computed the location of the annulus line) on the overlay may be performed by three different software processes, as described herein. Each software process may be implemented during different steps along the clinical procedure (e.g., TAVI procedure), for example: The first software process may be implemented for annulus line annotation following analysis of contrast agent injection, for example, as described with reference to 1612. The second software process may be implemented for annulus line annotation in reference to the position of the accessory catheter (e.g., pigtail), for example, as described with reference to 1632. The third software process may be implemented for annulus line annotation in reference to the position of synchronized structures, for example, calcification signature, for example, as described with reference to 1636. The same validation may be used for each of the three aforementioned software processes. The location of block 1650 after 1632 in the flow of FIG. 14 is meant as one example, and may be located in other parts of the flow such as after 1612 and/or after 1636.

As part of the validation process, a fluoroscopy sequence (CINE) of the contrast injection, which includes the automatic annotations of the annulus line (i.e. overlay indicating the location of the annulus line on the fluoroscopic image), may be provided.

Referring now back to FIG. 18, fluoroscopic image 2002 including the overlay of two dashed parallel lines 2004 and 2006, is shown. Lines 2004 and 2006 are located with a clinical margin of for example, about 1 mm, or about 1.5 mm, or 2 mm, or other values, from an annulus line 2008, computed as described herein. Fluoroscopic image 2002 may be contrast or non-contrast, as described herein. A user may review these CINEs and determine whether the dashed annotations have been accurately positioned to include the actual annulus line. The validation process may align with the actual procedural workflow, wherein the physician may approve the position of the annulus line as an integral step in the procedure and make necessary adjustments if deemed necessary. Validation of accuracy of computing the annulus line may be determined when a minimum agreement between the automatically generated line and the user indicating correctness of the location of the automatically generated line is above a threshold, for example, above about 75%, or 85%, or 90%, or other values.

Referring now back to FIG. 14, the method may iterate back from 1602, for dynamically updating the overlay for tracking the location of the annulus line. A display presenting the overlay to the user may be dynamically updated.

The location of the annulus line may be dynamically tracked on subsequent images without contrast. The tracking may be done, by mapping the location of the annulus line detected on a baseline image without contrast to a subsequent image without contrast, based on a registration between the baseline image and subsequent image. For example, computing optical flow between the baseline image and the subsequent image, and applying the optical flow to map the annulus line from the baseline image to the subsequent image. The mapping from the baseline image to the subsequent image may be computationally efficient in comparison to re-computing the combined mapping for each new image, which may enable real-time tracking.

Optionally, the annulus line is tracked prior to an event that disrupts the annulus, for example, inflation of a balloon within the native valve (e.g., valvuloplasty) such as to crack calcifications and/or adhesions to increase the area of the native valve to enable deployment of the prosthetic heart valve. The annulus line may be detected after the event, using the spatial relationship computed prior to the event. The annulus line may be detected after the event using the same spatial relationship computed prior to the event, since the event disrupts the leaflets and/or calcifications adhering to the leaflets and surrounding tissues, but the annulus line remains substantially unaffected.

The descriptions of the various embodiments of the present invention have been described in reference to a trans-catheter aortic valve replacement intervention; however, the embodiments of the present invention are not limited to such intervention and may include any trans-catheter valve replacement intervention; e.g., tricuspid valve, mitral valve and pulmonary valve replacement intervention.

As used herein, the term aortic valve prosthesis device represents a not necessarily limiting and/or exemplary implementation. Other medical devices (e.g., other valve prosthesis device) which are inserted via a trans-catheter procedure into lumen of the body may be used.

The descriptions of the various embodiments of the present invention have been described in reference to template of calcification signature of the subject; this is a not necessarily limiting and/or exemplary implementation. Other anatomical structure (e.g., specific-subject anatomical structures) may be used, for example: tissue thickening and/or blood flow derived dynamic changes.

The descriptions of the various embodiments of the present invention have been described in reference to obtaining an indication of the annulus line of the heart of the subject; this is a not necessarily limiting and/or exemplary implementation. Other structures within a native valve of a subject may be used as well; for example: middle of a subject's native aortic valve, top of a subject's native aortic valve, one or more commissures of a subject's native aortic valve, the leaflets of a subject's native aortic valve and the sinuses next to a subject's native aortic valve.

The descriptions of the various embodiments of the present invention have been described in reference to CT or MRI images taken prior to the procedure; however, the embodiments of the present invention are not limited to such imaging techniques and may include any imaging techniques; e.g., angiography, ultrasound and Doppler.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant ML models will be developed and the scope of the term ML model is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A computer implemented method of generating an image for guiding a trans-catheter aortic valve replacement intervention in a subject, comprising:
obtaining at least one fluoroscopic image depicting an aortic valve prosthesis device in the subject, the at least one fluoroscopic image excludes injected contrast,
wherein the at least one fluoroscopic image is a two-dimensional (2D) image;
feeding the at least one fluoroscopic image into a machine leaning (ML) model configured for processing 2D fluoroscopic images;
obtaining an indication of an annulus line of the heart of the subject from the ML model, wherein the annulus line comprises a virtual annulus line on the at least one fluoroscopic image corresponding to a location of a real annulus line in the subject, wherein the real annulus line cannot be seen and/or cannot be accurately seen in the at least one fluoroscopic image that excludes injected contrast; and
presenting the indication.

2. The computer implemented method of claim 1,
wherein the at least one fluoroscopic image comprises at least one non-contrast image depicting a structure associated with a valve; and further comprising:
obtaining at least one contrast image comprising at least one fluoroscopic image including injected contrast, depicting the annulus line of the heart, obtained at a same zoom, subject orientation, and pose of an image sensor that captured the non-contrast image;
feeding a combination of the at least one non-contrast image and the at one contrast image into the machine learning model;
wherein the indication of the annulus line is presented as a visual overlay over the at least one non-contrast image.

3. The computer implemented method of claim 2, wherein the at least one non-contrast image and the at least one contrast image depict a same fiducial object.

4. The computer implemented method of claim 3, wherein the fiducial object comprises a catheter located in the aorta of the subject.

5. The computer implemented method of claim 4, wherein the machine learning model is trained on a training dataset of multiple records, each record includes a combination of at least one non-contrast image that depicts the structure associated with the valve and a fiducial object, and at least one contrast image that depicts the annulus line of the heart of the subject and the fiducial object.

6. The computer implemented method of claim 2, further comprising detecting a change in at least one of the zoom, subject orientation, and the pose of the image sensor, and feeding the detected change to the machine learning model in combination with the non-contrast image.

7. The computer implemented method of claim 2, further comprising feeding pairs of sequentially obtained non-contrast images into the machine learning model for tracking a location of the annulus line.

8. The computer implemented method of claim 1, further comprising presenting the indication as a visual overlay over the at least one fluoroscopic image on a display.

9. The computer implemented method of claim, wherein the machine learning model is trained on a training dataset of fluoroscopic images that depict the annulus line of the heart of the subject, and a ground truth of the indication for the annulus line of the heart of the subject.

10. The computer implemented method of claim 1, wherein the obtaining the at least one fluoroscopic image, the feeding, the obtaining the indication, and the presenting, are iterated while the valve is transported on its way to be implanted in a native aortic annulus.

11. The computer implemented method of claim 1, further comprising processing the at least one fluoroscopic image by comparing the fluoroscopic angulation of the at least one fluoroscopic image to at least one image of a training dataset used to train the ML model and/or at least one image captured prior to the procedure that excludes the aortic valve prosthesis device.

12. The computer implemented method of claim 1, wherein the indication comprises at least one of: position of the annulus line of the heart of the subject and orientation of the annulus line of the heart of the subject.

13. The computer implemented method of claim 1, according further comprising extracting a plurality of landmarks from the at least one fluoroscopic image, and feeding the plurality of landmarks into the ML model to obtain the indication.

14. The computer implemented method according to claim 13, wherein the plurality of landmarks includes one of more of: anatomical structures around the annulus line and signature of calcification presence in the heart.

15. The computer implemented method of claim 1, wherein the ML model is a generic ML model, and further comprising dynamically updating the generic ML model to create a customized ML model for the subject by dynamically creating customized records that include fluoroscopic images of the subject, and dynamically training the generic ML model on the customized records.

16. The computer implemented method of claim 15, wherein the customized records that include fluoroscopic images of the subject were taken when the subject was injected with contrast.

17. The computer implemented method of claim 15, wherein the customized records include a first set of fluoroscopic images of the subject with contrast and a second set of fluoroscopic images without contrast, wherein ground truth labels for a target anatomical feature are applied to the customized records with the first set of fluoroscopic images with contrast.

18-26. (canceled)

27. A system for generating an image for guiding a trans-catheter aortic valve replacement intervention in a subject, comprising:
at least one processor executing a code for:
obtaining at least one fluoroscopic image depicting an aortic valve prosthesis device in the subject, the at least one fluoroscopic image excludes injected contrast,
wherein the at least one fluoroscopic image is a two-dimensional (2D) image;
feeding the at least one fluoroscopic image into a machine leaning model configured for processing 2D fluoroscopic images;
obtaining an indication of an annulus line of the heart of the subject from the ML model, wherein the annulus line comprises a virtual annulus line on the at least one fluoroscopic image corresponding to a location of a real annulus line in the subject, wherein the real annulus line cannot be seen and/or cannot be accurately seen in the at least one fluoroscopic image that excludes injected contrast; and
presenting the indication as a visual overlay over the at least one fluoroscopic image on a display.

28-148. (canceled)

149. The computer implemented method of claim 1, wherein the annulus line is defined as a line intersecting nadirs of at least two cusps of an aortic valve.
